(19) United States
(12) Patent Application Publication
(10) Pub. No.: US 2008/0082109 A1
(43) Pub. Date: Apr. 3, 2008

(54) ROBOTIC SURGICAL SYSTEM WITH FORWARD-ORIENTED FIELD OF VIEW GUIDE INSTRUMENT NAVIGATION

(75) Inventors: Frederic H. Moll, Woodside, CA (US); Federico Barbagli, San Francisco, CA (US); Christopher R. Carlson, Menlo Park, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 11/852,255

(22) Filed: Sep. 7, 2007

Related U.S. Application Data
(63) Continuation-in-part of application No. 11/829,076, filed on Jul. 26, 2007.
(63) Continuation-in-part of application No. 11/833,969, filed on Aug. 3, 2007.
(60) Provisional application No. 60/843,274, filed on Sep. 8, 2006.

(51) Int. Cl. A61B 19/00

(52) U.S. Cl. 606/130

(57) ABSTRACT

A robotic surgical system includes an instrument driver and an instrument assembly operatively coupled to the instrument driver such that mechanisms of the instrument driver operate or control movement, operation, or both, of components of the instrument assembly. The instrument assembly components include an elongate flexible guide instrument and an image capture device, wherein the image capture device is configured to capture images of a forward-oriented field of view. The system further comprises a controller operatively coupled to the instrument driver and configured to operate the instrument driver mechanisms in a manner so as to control advancement of the instrument assembly toward a target along a trajectory that maintains the target in the forward-oriented field of view of the image capture device.

### RELATED APPLICATION DATA

The present application is a continuation-in-part of U.S. application Ser. No. 11/829,076, filed Jul. 26, 2007, and a continuation-in-part of U.S. application Ser. No. 11/833,969, filed Aug. 3, 2007. The present application also claims the benefit under 35 U.S.C. § 119 to U.S. Provisional Patent Application Ser. No. 60/843,274, filed Sep. 8, 2006. The foregoing applications are all incorporated by reference into the present application in their entirety for all purposes.

### FIELD OF INVENTION

The invention relates generally to robotically controlled systems, such as telerobotic surgical systems, and more particularly to robotic catheter systems for performing minimally invasive diagnostic and therapeutic procedures.

### BACKGROUND

Robotic diagnostic and interventional systems and devices are well suited for use in performing minimally invasive medical procedures, as opposed to conventional techniques wherein a patient's body cavity is open to permit the surgeon's hands access to the internal organs. There is a need for highly controllable yet minimally sized systems to facilitate imaging, diagnosis, and treatment of tissues which may lie deeply and/or concealed within the body cavity of a patient, and which may be accessed through natural body orifices or percutaneous incisions and by way of naturally-occurring pathways such as blood vessels or other bodily lumens.

### SUMMARY OF THE INVENTION

In accordance with various embodiments of the present invention, a robotic surgical system includes an instrument driver, and an instrument assembly operatively coupled to the instrument driver, e.g., via a remote communication link, such that mechanisms of the instrument driver operate or control movement, operation, or both, of components of the instrument assembly. The instrument assembly components including an elongate flexible guide instrument and an image capture device, wherein the image capture device is configured to capture images of a forward-oriented field of view. The system further comprises a controller operatively coupled to the instrument driver and configured to operate the instrument driver mechanisms in a manner so as to control advancement of the instrument assembly toward a target along a trajectory that maintains the target in the forward-oriented field of view of the image capture device.

In one embodiment, the controller utilizes a software-implemented orientation platform (e.g., a Stewart or Gough platform) to maintain the target in the forward-oriented field of view of the image capture device. In one embodiment, the controller utilizes a software-implemented receding-horizon control algorithm that provides outputs for operating the instrument driver mechanisms to maintain the target in the forward-oriented field of view of the image capture device. In one embodiment, the controller utilizes a software-implemented pattern recognition algorithm for identifying target objects or target features in images acquired by the image capture device and providing outputs for operating the instrument driver mechanisms to maintain the identified target objects or target features in the forward-oriented field of view of the image capture device.

In various embodiments, the controller is configured to position or orient the elongate flexible guide instrument using discounted tangent adjustments in order to maintain the target in the forward-oriented field of view of the image capture device. In various embodiments, the system comprises a monitor for displaying images of the forward-oriented field of view acquired by the image capture device, and a user input device coupled to the controller for controlling movement, operation, or both, of the components of the instrument assembly wherein movement of the user input device is calibrated with the elongate flexible guide instrument such that a directional input to the user input device produces a corresponding directional movement of the forward-oriented field of view displayed on the monitor. In one embodiment, the controller is operatively coupled to the display and configured to supply an indicated image of a working tool on the display when the working tool is outside of the forward-oriented field of view.

In some embodiments, the robotic surgical system further comprises a working tool (e.g., a laser fiber, a gripper, or a basket) operatively coupled to the instrument assembly and configured to be independently navigated relative to the guide instrument.

In some embodiments, the image capture device includes a fish-eye type lens for capturing or presenting selected sectors of the forward-oriented field of view.

Other embodiment, aspects, and advantages of the present invention will become apparent from the following description, taken in conjunction with the accompanying drawings.

### BRIEF DESCRIPTION OF THE DRAWINGS

The present invention will be readily understood by the following detailed description, taken in conjunction with accompanying drawings, illustrating by way of examples the principles of the invention. The drawings illustrate the design and utility of preferred embodiments of the present invention, in which like elements are referred to by like reference symbols or numerals. The objects and elements in the drawings are not necessarily drawn to scale, proportion, or precise positional relationships; instead emphasis is focused on illustrating the principles of the invention.

FIG. 1 illustrates one embodiment of a robotic surgical system.

FIG. 2 illustrates another embodiment of a robotic surgical system.

FIG. 3 illustrates one embodiment of a robotic surgical system being used to perform diagnostic and/or interventional operations on a patient.

FIG. 4A illustrates a cross sectional view of a heart.

FIG. 4B illustrates an instrument assembly advanced into a chamber of the heart.

FIG. 4C illustrates an ablation tool advanced through the lumen of the instrument assembly into a chamber of the heart.

FIG. 5A illustrates a target of an operation site in a chamber of the heart.

FIG. 5B illustrates an instrument assembly advanced toward a target site in a chamber of the heart.

FIG. 5C illustrates an ablation tool advanced through a lumen of an instrument assembly toward a target site in a chamber of the heart.

FIG. 6A through 6C respectively illustrate an instrument assembly and an ablation tool being used to address a target site related to atrioventricular nodal reentrant tachycardia.

FIG. 7A through FIG. 7C respectively illustrates an instrument assembly and an ablation tool being used to address a target site related to ventricular tachycardia.

FIG. 7D through FIG. 7F respectively illustrates an instrument assembly being used to address a target site related to a left-sided ventricular tachycardia.

FIG. 7G through FIG. 7I respectively illustrates a retrograde approach to address a ventricular tachycardia condition.

FIG. 8A illustrates an instrument assembly being used to treat a patent foramen ovale condition.

FIG. 8B illustrates an instrument assembly with an ablation tool being used to treat a patent foramen ovale condition.

FIG. 8C and FIG. 8D respectively illustrates an instrument assembly with a suturing tool being used to treat a patent foramen ovale condition.

FIG. 8E and FIG. 8F respectively illustrates an instrument assembly with a clip application tool being used to treat a patent foramen ovale condition.

FIG. 8G and FIG. 8H respectively illustrates an instrument assembly with a needle instrument being used to treat a patent foramen ovale condition.

FIG. 8I and FIG. 8J respectively illustrates an instrument assembly with an irritation tool being used to treat a patent foramen ovale condition.

FIG. 9A and FIG. 9B respectively illustrates an instrument assembly with a suturing tool being used to treat a left atrial appendage occlusion condition.

FIG. 9C through FIG. 9H respectively illustrates an instrument assembly coupled with various tools being used to treat a left atrial appendage occlusion condition.

FIG. 10A and FIG. 10B respectively illustrates an instrument assembly with lead deploying tool.

FIG. 10C and FIG. 10D respectively illustrates an instrument assembly deploying leads in the right and left atrium of the heart.

FIG. 11A through FIG. 11F respectively illustrates an instrument assembly with various tools being used to treat a chronic total occlusion condition.

FIG. 12A and FIG. 12B respectively illustrates an instrument assembly with an injection tool being used to treat congestive heart failure condition.

FIG. 12C illustrates one embodiment of an injection pattern for treating infracted tissue.

FIG. 13A through FIG. 13G respectively illustrates an instrument assembly with various tools being used to perform valve repair procedures.

FIG. 13H and FIG. 13I illustrate the chords, chordae tendineae, or papillary muscle of the mitral valve leaflet being adjusted.

FIG. 14 illustrates an instrument assembly with an ablation tool being used to perform valve repair.

FIG. 15A through FIG. 15D illustrate a retrograde method to deploy an expandable aortic valve prosthetic to repair an aortic valve.

FIG. 15E through FIG. 15J illustrate a method of deploying an expandable valve prosthetic by way of the inferior vena cava through the septum and the mitral valve to the aortic valve.

FIG. 15K illustrates a two-handed approach to deploy an expandable valve prosthetic.

FIG. 16 illustrates an instrument assembly with a lithotripsy laser fiber for performing lithotripsy procedures.

FIG. 17 illustrates an instrument assembly with a grasper including an energy source configured for performing lithotripsy procedures.

FIG. 18 illustrates an instrument assembly with a basket tool including an energy source configured for performing lithotripsy procedures.

FIG. 19 illustrates an expandable grasping tool assembly including an energy source.

FIG. 20 illustrates a bipolar electrode grasper assembly.

FIG. 21 illustrates an instrument assembly configured with basket arms.

FIG. 22 illustrates an instrument assembly including a lithotripsy fiber and image capture device.

FIG. 23 illustrates an instrument assembly including a grasping tool.

FIG. 24 illustrates an instrument assembly including a basket tool apparatus.

FIG. 25 and FIG. 26 respectively illustrates an operation of an instrument assembly with a basket tool apparatus.

FIG. 27 illustrates an instrument assembly including a basket arm capture device and image capture device.

FIG. 28 illustrates an instrument assembly including a balloon apparatus.

FIG. 29 illustrates an instrument assembly including another balloon apparatus.

FIG. 30 illustrates an instrument assembly including yet another balloon apparatus.

FIG. 31 through FIG. 33 respectively illustrates an instrument assembly including an inflatable balloon cuff apparatus.

FIG. 34 through FIG. 36 respectively illustrate an instrument assembly including a flexible balloon cuff apparatus.

FIG. 37 and FIG. 38 respectively illustrates an instrument assembly including image capture apparatuses.

FIG. 39 through FIG. 40 respectively illustrates detailed views of the image capture assembly.

FIG. 41 illustrates a cross sectional view of a tubular structure for housing the image capture device assembly.

FIG. 42 through FIG. 45 respectively illustrates variations of embodiments of image capture assembly.

FIG. 46A illustrates a steerable instrument assembly being used in the bladder.

FIG. 46B illustrates a steerable instrument assembly being used in the prostate.

FIG. 47 illustrates another steerable instrument assembly.

FIG. 48 and FIG. 49 respectively illustrates yet another steerable instrument assembly.

FIG. 50A illustrates an instrument assembly being navigated toward a target.

FIG. 50B illustrates an instrument assembly having been navigated toward a target.

FIG. 51 illustrates a plot of various positions of an instrument assembly along a manifold curve as it is being navigated toward a target.

FIG. 52A illustrates one embodiment of a Stewart or Gough platform.

FIG. 52B illustrates another embodiment of a Stewart or Gough platform.

FIG. 53A illustrates an initial field of view before a pattern recognition technique is applied.

FIG. 53B illustrates a subsequent field of view after a pattern recognition technique is applied.

FIG. 54A illustrates another initial field of view before a pattern recognition technique is applied.

FIG. 54B illustrates a subsequent field of view after a pattern recognition technique is applied.

FIG. 55A through FIG. 55C illustrate some of the calibration processes of the input device and field of view.

FIG. 56A illustrates one image of a field of view.

FIG. 56B illustrates one desired image of a field of view with an indication of a tool that is outside of the field of view.

### DETAILED DESCRIPTION OF THE ILLUSTRATED EMBODIMENTS

Reference will now be made in detail to the preferred embodiments of the present invention, examples of which are illustrated in the accompanying drawings. While the invention will be described in conjunction with the preferred embodiments, it will be understood that they are not intended to limit the invention to these embodiments. On the contrary, the invention is intended to cover modifications, alternatives, and equivalents that may be included within the spirit and scope of the invention as defined by the appended claims. Furthermore, in the following detailed description of the embodiments, numerous specific details are set forth in order to provide a thorough understanding of the present invention. However, it will be readily apparent to one skilled in the art that the present invention may be practiced without these specific details. In other instances, well-known methods, procedures, and components have not been described in detail so as not to unnecessarily obscure aspects of the present invention.

Standard surgical procedures typically involve using a scalpel to create an opening of sufficient size to enable a surgical team to gain access to an area in the body of a patient for the surgical team to diagnose and treat one or more target sites. When possible, minimally invasive surgical procedures may be used instead of standard surgical procedures to minimize physical trauma to the patient and reduce recovery time for the patient to recuperate from the surgical procedures. Minimally invasive surgical procedures typically require using extension tools (e.g., catheters, etc.) to approach and address the target site through natural pathways (e.g., blood vessels, gastrointestinal tract, etc.) from a remote location either through a natural body orifice or a percutaneous incision. As can be appreciated, the surgeon may have limited information or feedback (e.g., visual, tactile, etc.) to accurately navigate the extension tools, such as one or more catheters, and place the working portions of the extension tools at precise locations to perform the necessary diagnostic and/or interventional procedures. Even with such potential limitations, minimally invasive surgical procedures may be more effective and beneficial for treating the patient, instead of standard open surgery.

Minimally invasive diagnostic and interventional operations may require the surgeon to remotely approach and address the operation or target site by using extension tools. The surgeon usually approaches the target site through either a natural body orifice or a small percutaneous incision in the body of the patient. In some situations, the surgeon may use multiple extension tools and approach the target site through one or more natural body orifices as well as small percutaneous incisions in the body of the patient. Typically, the natural body orifices or small incisions are located at some distance away from the target site. Extension tools (e.g., various types of catheters and surgical instruments) enter the body through one or more natural body orifices or small percutaneous incisions, and the extension tools are guided, navigated, manipulated, maneuvered, and advanced toward the target site typically by way of natural body pathways (e.g., blood vessels, esophagus, trachea, small intestine, large intestine, urethra, etc.). The extension tools might include one or more catheters as well as other surgical tools or instruments. The catheters may be manually controlled catheters or robotically operated catheters. In most situations, the surgeon has limited visual and tactile information to discern the location of the catheters and surgical instruments relative to the target site and/or other organs in the patient.

For example, in the treatment of cardiac arrhythmias such as atrial fibrillation (AF), cardiac ablation therapy is applied to the left atrium of the heart to restore normal heart function. For this operation, one or more catheters (e.g., sheath catheter, guide catheter, ablation catheter, endoscopic catheter, intracardiac echocardiography catheter, etc.) may be inserted through one or more natural orifices or one or more percutaneous incisions at the femoral vein near the thigh or pelvic region of the patient, which is located at some distance away from the operation or target site. In this example, the operation or target site for performing cardiac ablation is in the left atrium of the heart. Catheters may be guided (e.g., by a guide wire, a sheath, etc.), manipulated, maneuvered, and advanced toward the target site by way of the femoral vein to the inferior vena cava into the right atrium of the heart and through the interatrial septum to the left atrium of the heart. The catheters may be used separately or in combination of multiple catheters. Currently, the surgeon has limited visual and tactile information to assist him or her with maneuvering and controlling the catheters (separately or in combination). In particular, because of limited information and/or feedback, it is especially difficult for the surgeon to maneuver and control one or more distal portions of the catheters to perform cardiac ablation at precise locations or spots on the surface or wall of the left atrium of the heart. As will be explained below, embodiments of the present invention provide improved systems and methods that would facilitate imaging, diagnosis, address, and treatment of tissues which may lie deeply and/or concealed under other tissues or organs within the body cavity of a patient. With embodiments of the present invention, the surgeon may be able to position the catheter more precisely and accurately to address the operation or target sites. For example, with the improved imaging capability, the surgeon may be able to apply cardiac ablation at the desired locations or spots on the surface or wall of the left atrium of the heart in a more precise and accurate manner to address cardiac arrhythmias such as atrial fibrillation. In addition, U.S. patent application Ser. No. 11/185,432, filed on Jul. 19, 2005; U.S. patent application Ser. No. 11/202,925, filed on Aug. 12, 2005; and U.S. patent application Ser. No. 11/481,433, filed Jul. 3, 2006 are incorporated herein by reference in their entirety.

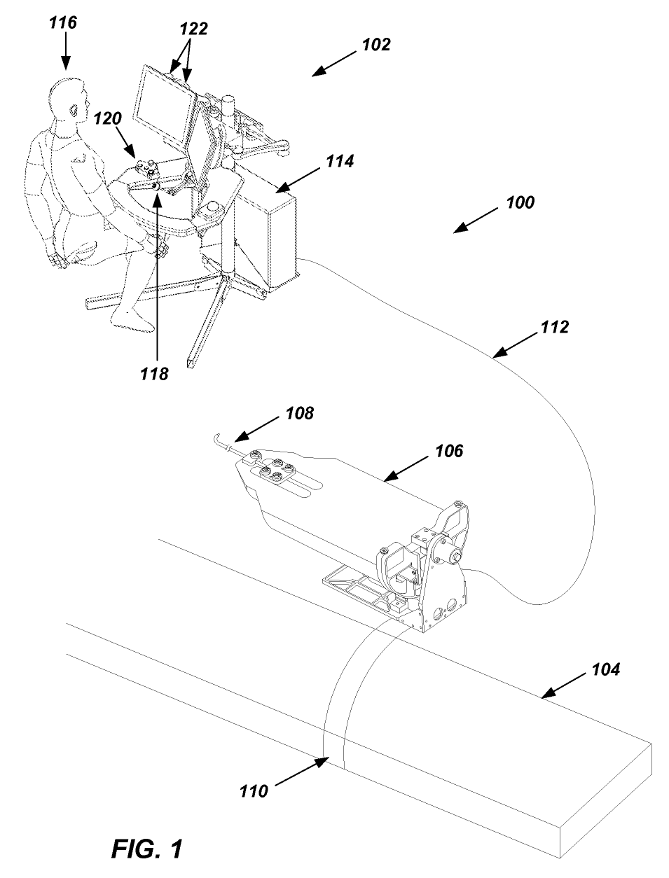

FIG. 1 illustrates one embodiment of a robotic surgical system (100), e.g., the Sensei™ Robotic Catheter System from Hansen Medical, Inc. in Mountain View, Calif., U.S.A., an operator control station (102) located remotely from an operating table (104) to which an instrument driver (106) and instrument assembly (108), e.g., the Artisan™ Control Catheter also from Hansen Medical, Inc. in Mountain View, Calif., U.S.A., are supported by an instrument driver mounting brace (110) that is mounted on the operating table (104). A wired connection (112) transfers signals between an electronics rack (114) at the operator control station (102) and instrument driver (106). The electronics rack (114) includes system hardware, software, firmware, and combinations thereof that substantially operate and perform the many functions of the robotic surgical system (100). The instrument driver mounting brace (110) is a substantially arcuate-shaped structural member configured to position the instrument driver (106) above a patient (not shown) who is lying on the operating table (104). The wired connection (112) may transmit manipulation and control commands from an operator or surgeon (116) who is working at the operator control station (102) to the instrument driver (106) to operate the instrument assembly (108) to perform minimally invasive operations on the patient who is lying on the operating table (104). The surgeon (116) may provide manipulation and control commands using a master input device (MID) (118). In addition, the surgeon may provide inputs, commands, etc. by using one or more keyboards (120), trackball, mouse, etc. The wired connection (112) may also transmit information (e.g., visual views, tactile or force information, position, orientation, shape, localization, electrocardiogram, map, model, etc.) from the instrument assembly (108), the patient, and monitors (not shown in this figure) to the electronics rack (114) for providing the necessary information or feedback to the operator or surgeon (116) to facilitate monitoring of the instrument assembly (108), the patient, and one or more target sites for performing precise manipulation and control of the instrument (108) during the minimally invasive surgical procedure. The wired connection (112) may be a hard wire connection, such as an electrical wire configured to transmit electrical signals (e.g., digital signals, analog signals, etc.), an optical fiber configured to transmit optical signals, a wireless link configured to transmit various types of signals (e.g., RF signals, microwave signals, etc.), or any combinations of electrical wire, optical fiber, wireless link, etc. The information or feedback may be displayed on one or more monitors (122) at the operator control station (102).

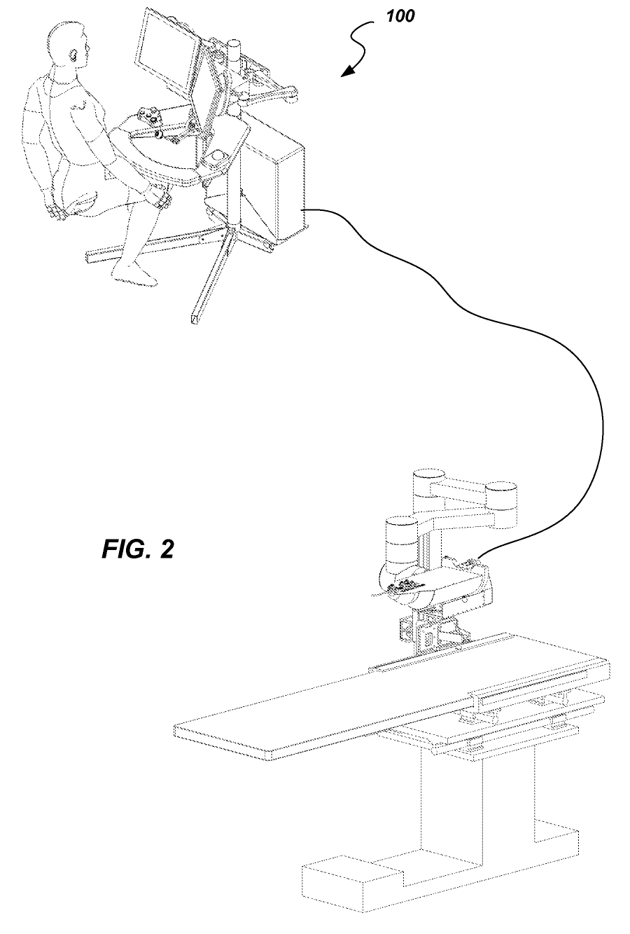

FIG. 2 illustrates another embodiment of a robotic surgical system (100). For more detailed discussions of robotic surgical systems, please refer to U.S. Provisional Patent Application No. 60/644,505, filed on Jan. 13, 2005; U.S. patent application Ser. No. 11/481,433, filed on Jul. 3, 2006; and U.S. patent application Ser. No. 11/637,951, filed on Dec. 11, 2006; and they are incorporated herein by reference in their entirety.

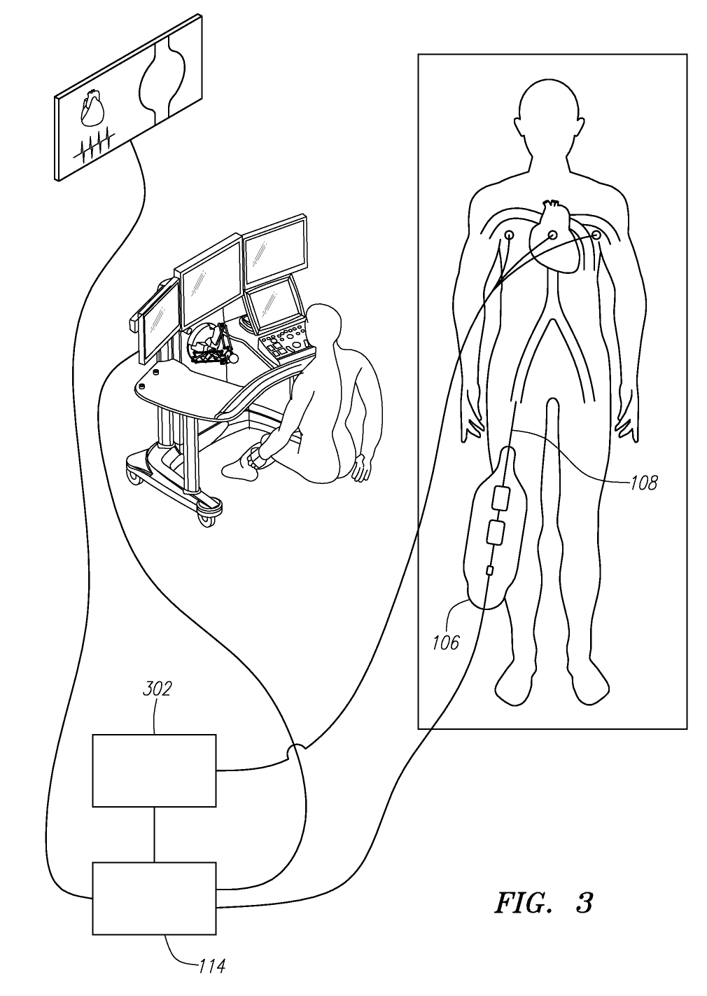

FIG. 3 illustrates one embodiment of a robotic surgical system (100) configured to perform minimally invasive surgery using one or more instrument assemblies (108). For example, the instrument assembly (108) may include a sheath catheter, guide catheter, ablation catheter, endoscopic catheter, intracardiac echocardiography catheter, etc., or any combination thereof. In addition, surgical instruments or tools (e.g., lasers, optics, cutters, needles, graspers, scissors, baskets, balloons, etc.) may be attached or coupled to any one or combination of the catheters. In one embodiment, the instrument assembly (108) may be a catheter system that includes a sheath catheter, guide catheter, a surgical catheter, and/or surgical instrument, such as the Artisan™ Control Catheter available from Hansen Medical, Inc. at Mountain View, Calif., U.S.A. The instrument assembly (108) also includes all the control mechanisms to operate its various components, e.g., sheath catheter, guide catheter, a surgical catheter, and/or surgical instrument. The robotic surgical system (100) including the control station (102), instrument driver (106), instrument (108), and the wired connection (112) may be used to treat or perform cardiac related diseases, maladies, conditions, or procedures (e.g., atrial flutter, Wolf-Parkinson-White (“WPW”), atrioventricular nodal reentrant tachycardia (“AVNRT”), Ventricular tachycardia (“V-tach”), patent foramen ovale (“PFO”), left atrial appendage occlusion, pacing lead placement, chronic total occlusion (“CTO”), ventricular injection therapy, valve repair).

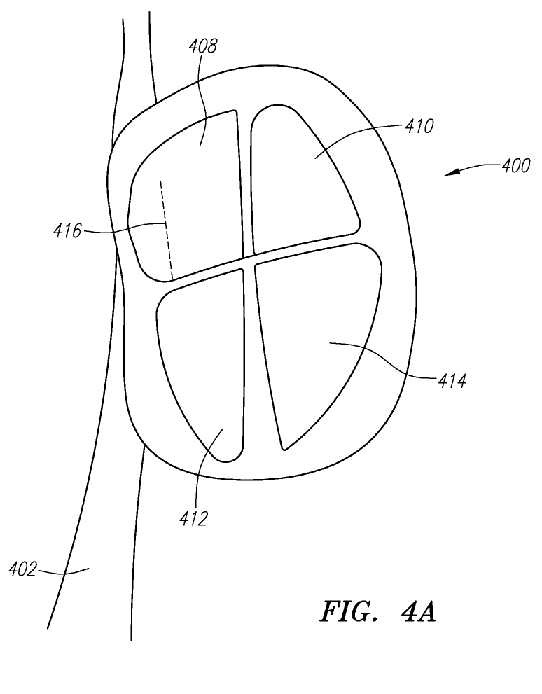

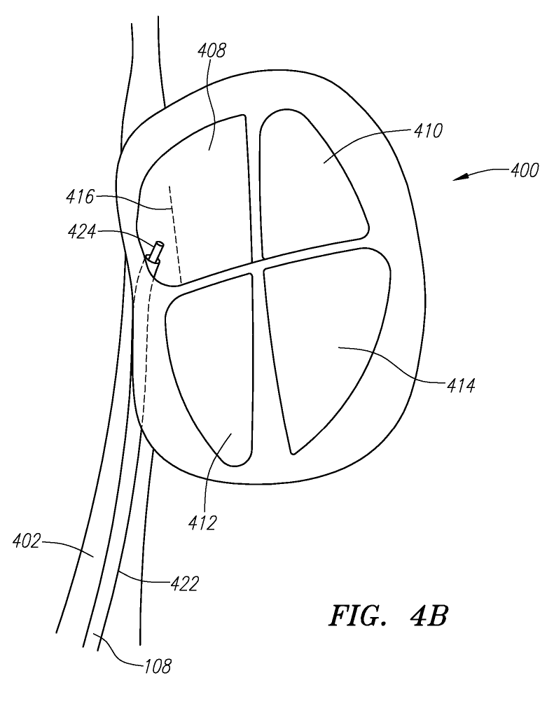

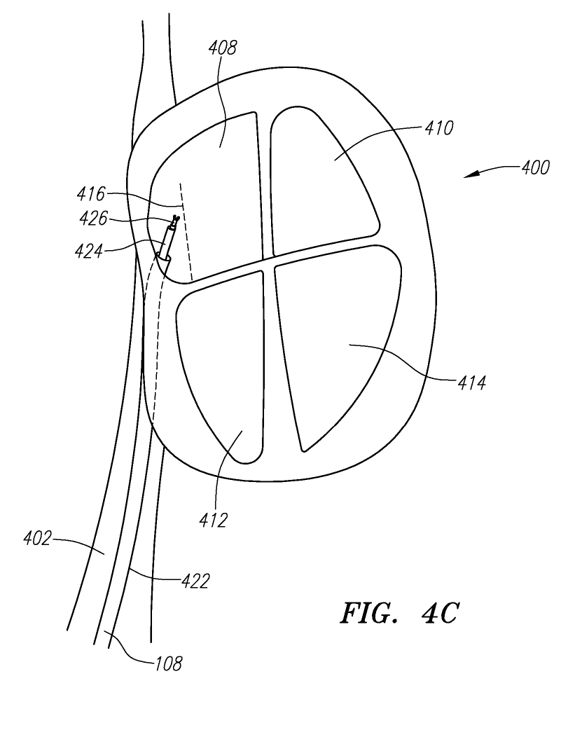

For example, atrial flutter is characterized by a rapid but organized and predictable pattern of beating of the atria. Similar to atrial fibrillation, the ventricles cannot respond to all of the atrial beats, which may cause blood to accumulate and collect or pool in the atria increasing the risk of stroke. FIG. 4A illustrates a cross sectional view of a heart (400). The cross sectional view illustrates the inferior vena cava (402), the right atrium (408), the left atrium (410), the right ventricle (412), and left ventricle (414). In addition, FIG. 4A illustrates a targeted location (416) (e.g., an area for linear lesion) for performing atrial flutter ablation lesion. FIG. 4B illustrates instrument (108) that may include a robotic sheath instrument or catheter (422) and a guide instrument or guide catheter (424) that have been navigated and positioned through the inferior vena cava (402) into the right atrium (408). Referring to FIG. 4C, an ablation tool (426) is depicted as having been navigated and placed through the working lumen of the guide instrument or guide catheter (424) and the ablation tool (426) is depicted as protruding slightly from the distal end of the guide instrument (424) to enable the guide instrument (424) to navigate the ablation tool (426) or the tip of the ablation tool (426) into position against portions of right atrium (408) to create the desired lesion (e.g., linear lesion), and preferably substantially treat or eliminate atrial flutter.

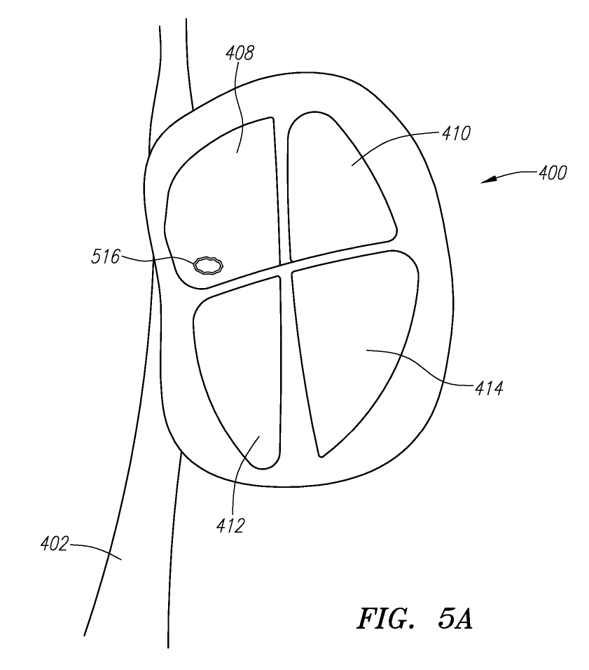

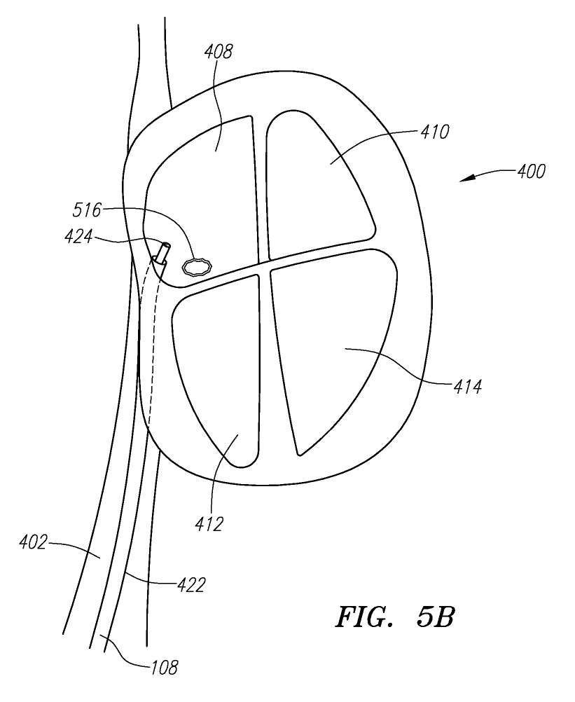

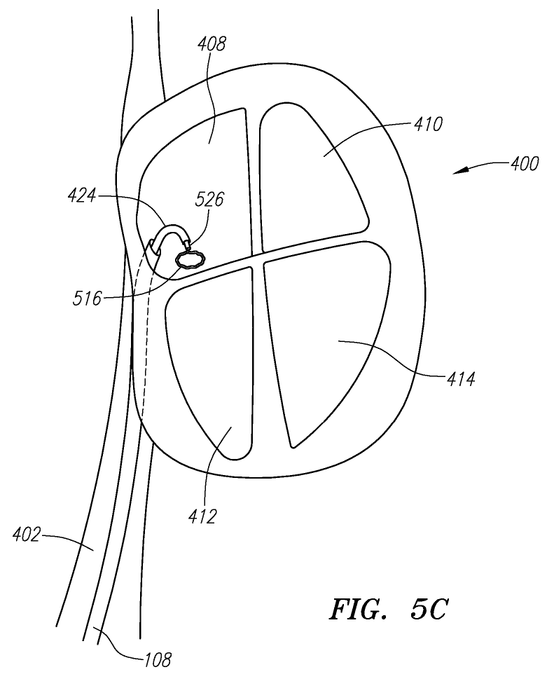

Wolf-Parkinson-White (“WPW”) is another type of arrhythmia that may be caused by an abnormal bridge of tissue, such as the eustachian ridge, which connects the atria and ventricles of the heart. This accessory pathway allows electrical signals to go back and forth between the atria and the ventricles without going through the heart's natural pacemaker, or atrioventricular node or AV node. If the signal ricochets back and forth, very fast heart rates and life-threatening arrhythmias can develop. Referring to FIG. 5A, an example of a targeted location (516) for an ablation lesion near or around the eustachian ridge is depicted. Referring to FIG. 5B, an instrument assembly (108) including a sheath instrument or sheath catheter (422) and a guide instrument or guide catheter (424) is depicted with the distal portions of the instruments (422 and 424) positioned in the right atrium (408). Referring to FIG. 5C, an ablation tool (526) is advanced through the working lumen or inner channel of the guide instrument (424) to a position wherein it may be utilized to contact and ablate desired portions of the targeted tissue.

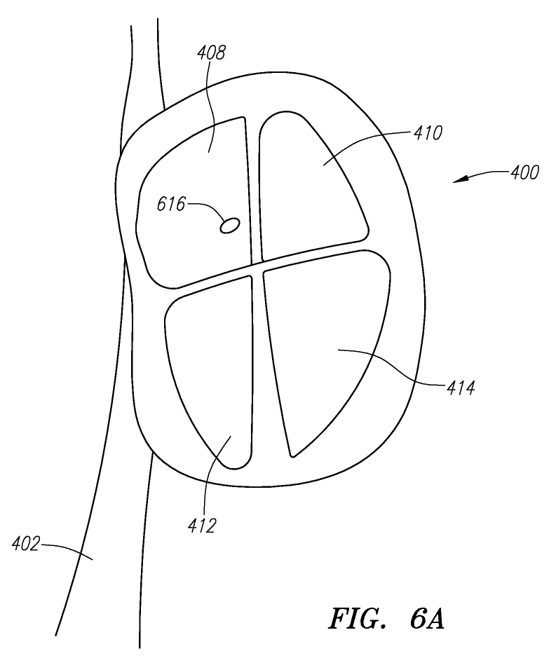

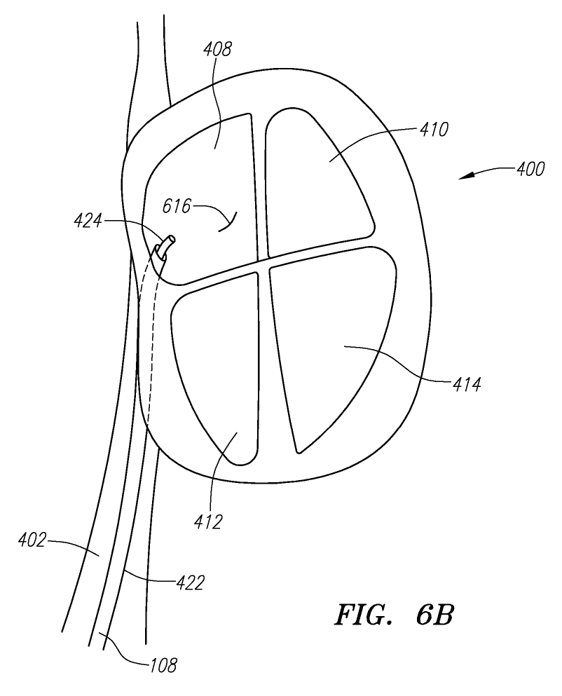

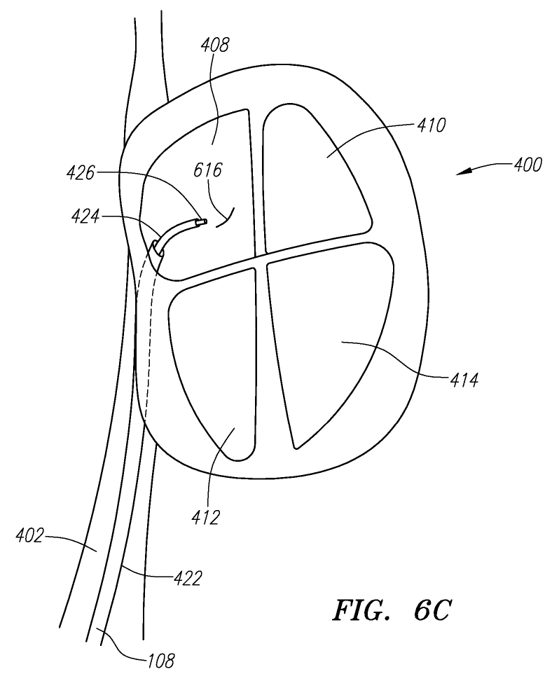

Atrioventricular Nodal Reentrant Tachycardia (“AVNRT”) is a common form of arrhythmia that arises from the atria. There are two distinct pathways between the atria and ventricle, one fast and one slow. In AVNRT, the abnormal signal begins in the atria and transfers to the AV node. Instead of conducting down to the ventricle, the signal is returned to the atria. Referring to FIGS. 6A-6C, a sheath (422) and guide (424) instrument assembly (108) may be utilized, along with an ablation catheter (626) or ablation electrode (626), to create an ablation lesion (616) in the right atrium (408) to address aberrant conduction pathways causing AVNRT.

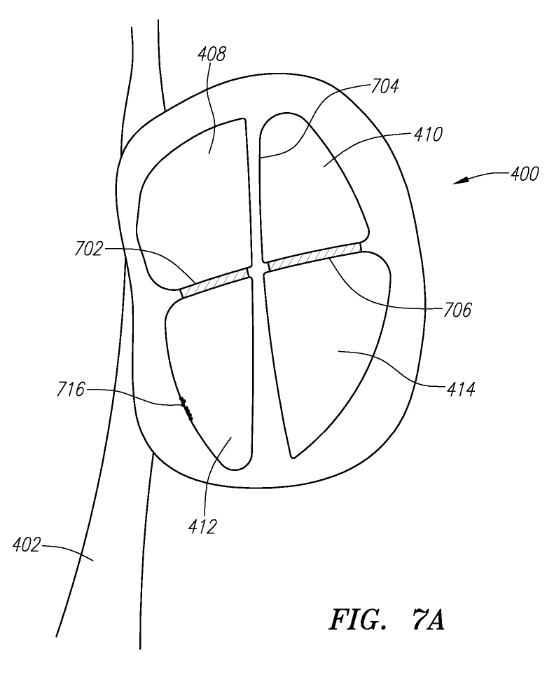

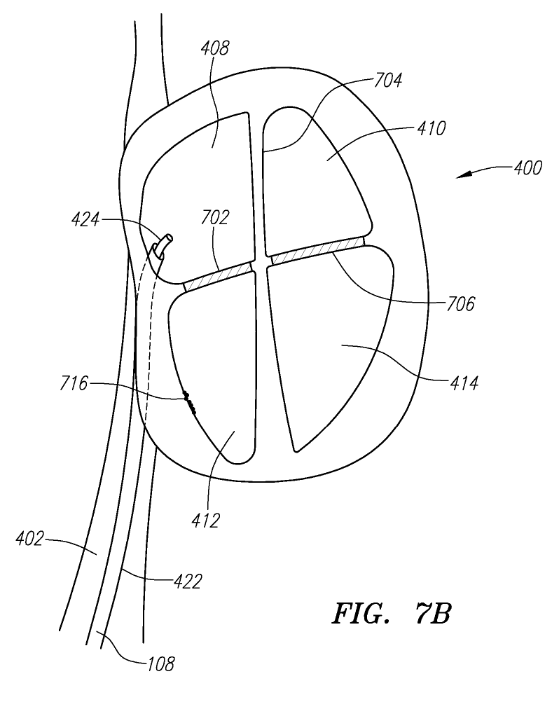

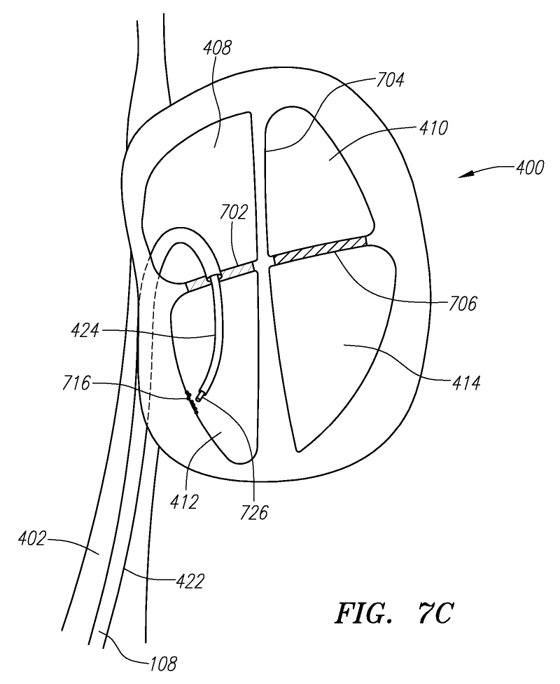

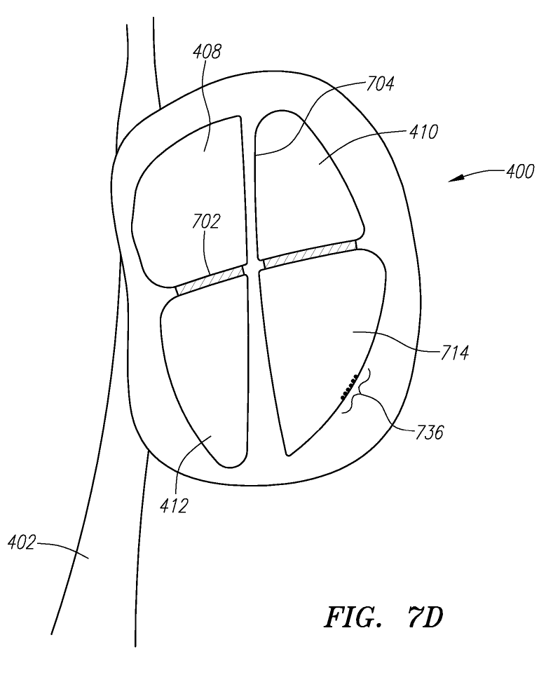

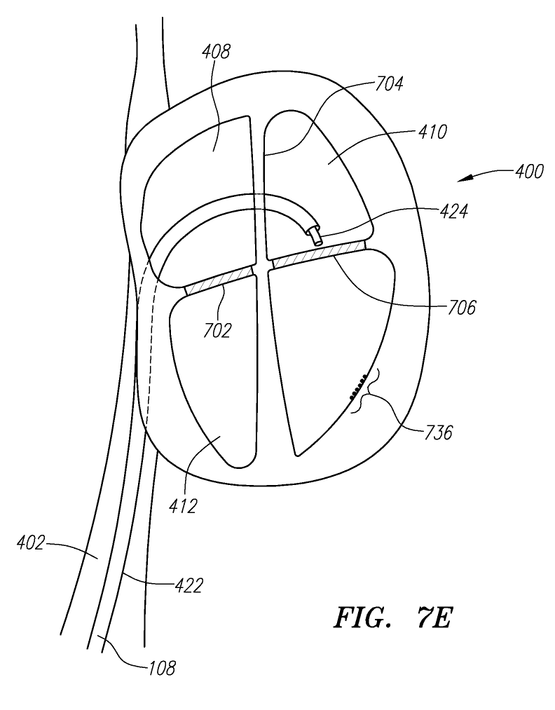

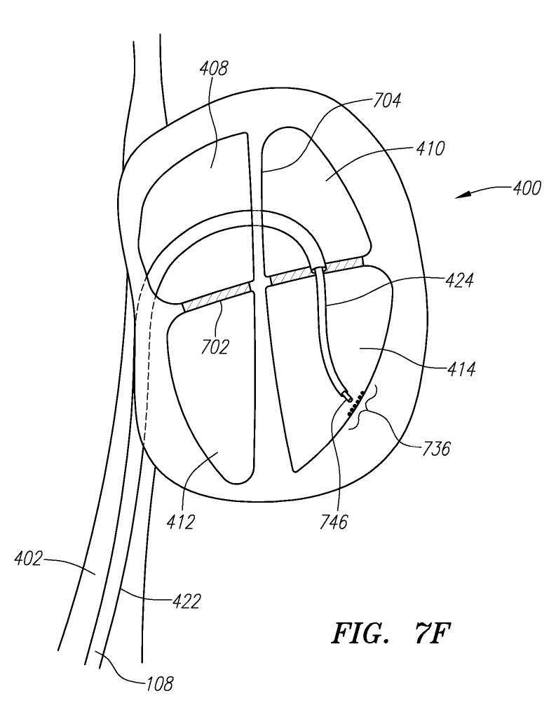

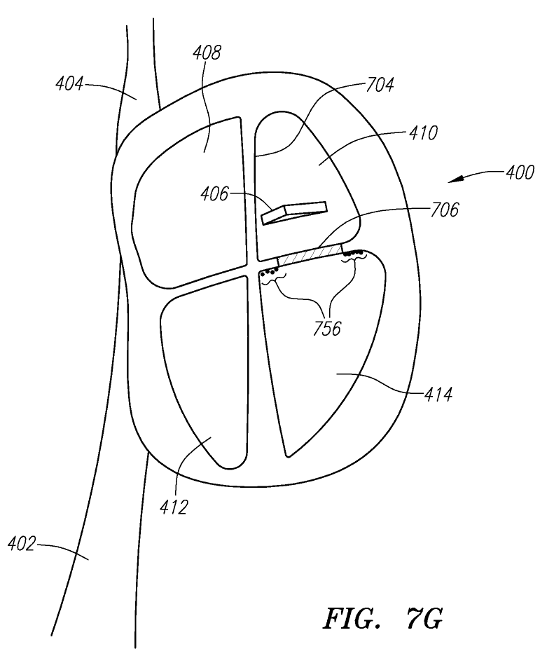

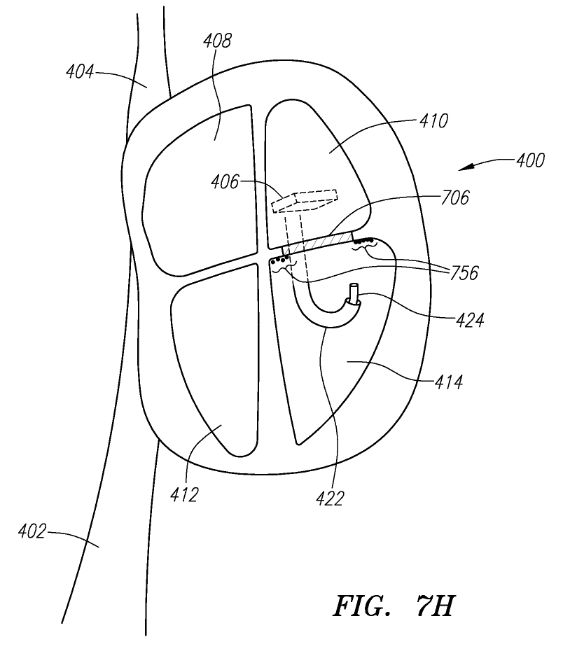

Ventricular tachycardia (“V-tach”) is a condition arises from the lower chambers of the heart as the name implies. It is characterized by heart rates over 100 beats per minute, but heart rates often approach 200 beats per minute. At this rate, very little blood is actually pumped out of the heart to the brain and other organs. As such, extremely fast V-tach can be fatal. Referring to FIGS. 7A-7C, a sheath (422) and guide (424) instrument assembly (108) may be utilized, along with an ablation catheter (726) or ablation electrode (726), to create an ablation lesion (716) in, for example, the right ventricle (412), to address aberrant conduction pathways causing right-sided V-tach. To reach the targeted lesion location, the sheath (422) may be positioned adjacent the tricuspid valve (702), and the guide (424) may be navigated across the tricuspid valve (702) to deliver the ablation electrode (726) against the targeted tissue, as depicted in FIG. 7C. FIGS. 7D-7F depict a similar instrument configuration (108) is utilized to address a left-sided V-tach scenario by navigating across the septum (704), by way of a transseptal puncture, into the left atrium (410), and down through the mitral valve (706) into the left ventricle (414) and to the targeted left ventricular tissue lesion (736) where an ablation lesion may be created to prevent aberrant conduction related to V-tach. FIGS. 7G-7I depict a retrograde approach, through the aorta (404), across the aortic valve (406), and into the left ventricle (414), subsequent to which the sheath instrument (422) may be utilized to direct the guide instrument (424) and ablation tool (766) up toward the inferior mitral annulus region (756) where ablation lesions may be created to address a V-tach scenario.

A patent foramen ovale (“PFO”) is an abnormal opening in the arterial septum which results in shunting of blood between the atrial chambers. PFOs are believed to be present in as many as 20% of the adult population and there is strong evidence that PFOs are responsible for the occurrence of a type of stroke, known as cryptogenic stroke, which occurs as a result of a blood clot in an otherwise healthy individual. Additionally, there is increasing evidence that the presence of a PFO is in some way related to the occurrence of migraine headache with aura in certain patients. Historically, PFOs have been treated with surgery, where the defect is sewn shut with direct suturing. Although this works well to close the defect, it requires open heart surgery and is very traumatic, which requires significant post-operative recovery. More recently, PFOs have been closed successfully with prosthetic patches that are delivered via a catheter based procedure. These procedures offer a minimally invasive approach, but require that the clinician leave prosthesis inside the heart to cover and occlude the PFO defect. The presence of foreign material inside the heart can lead to significant complications including infection, thrombus formation leading to stroke, development of cardiac arrhythmias, and dislodgment or migration of prosthesis that might necessitate surgical removal of the devices.

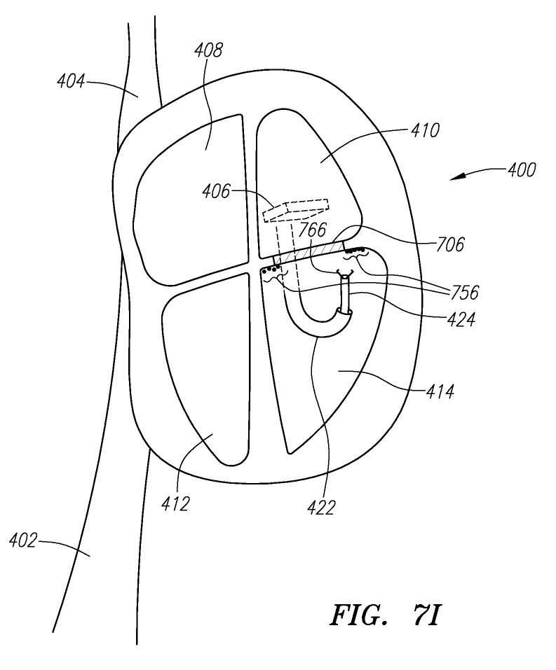

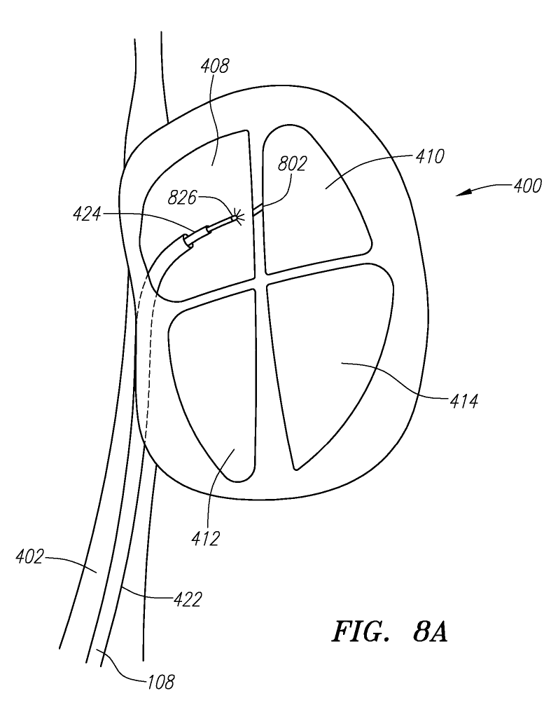

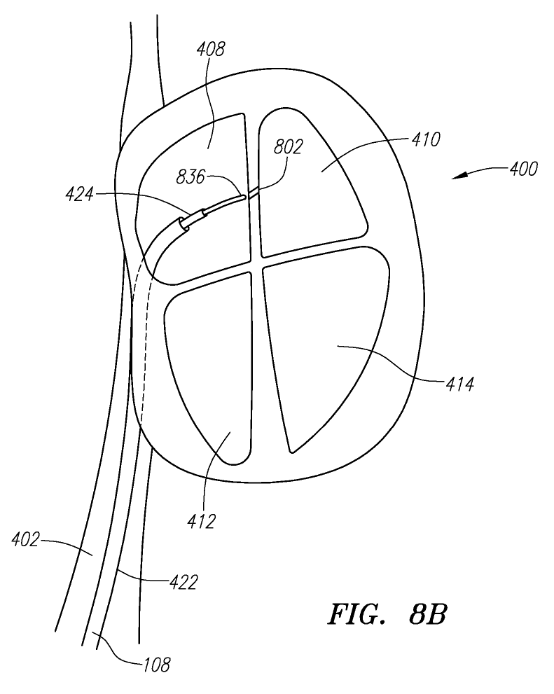

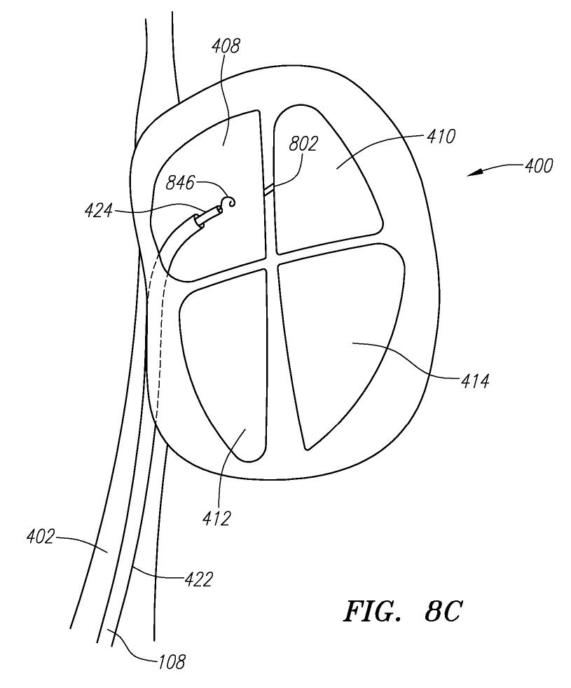

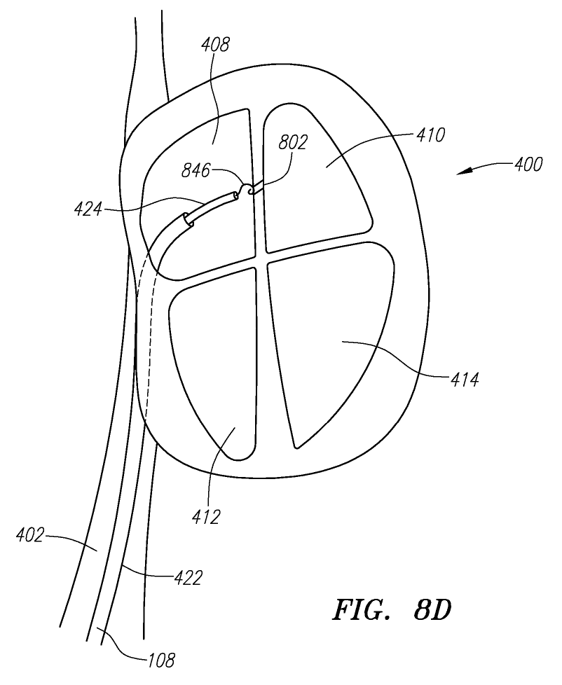

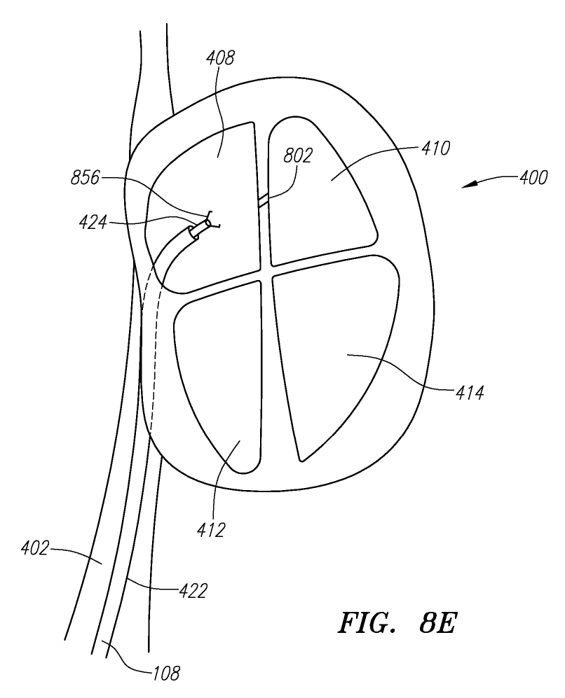

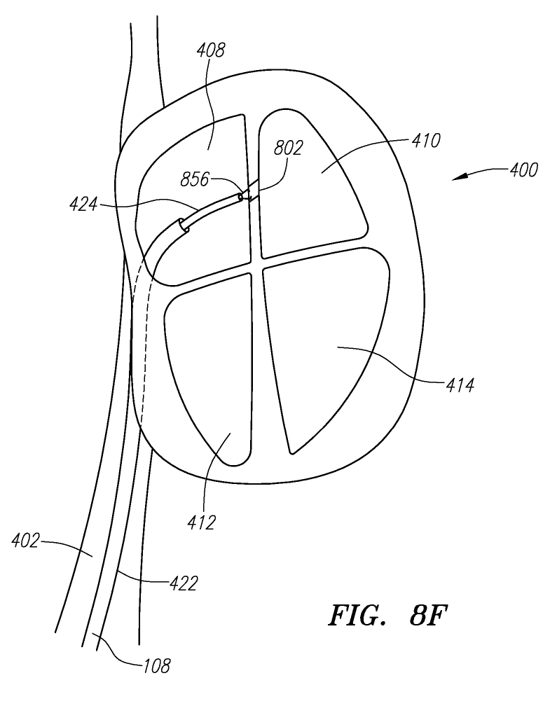

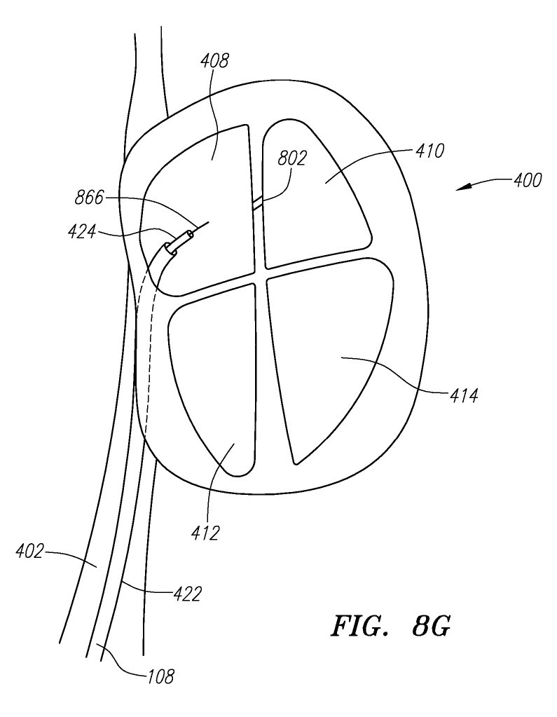

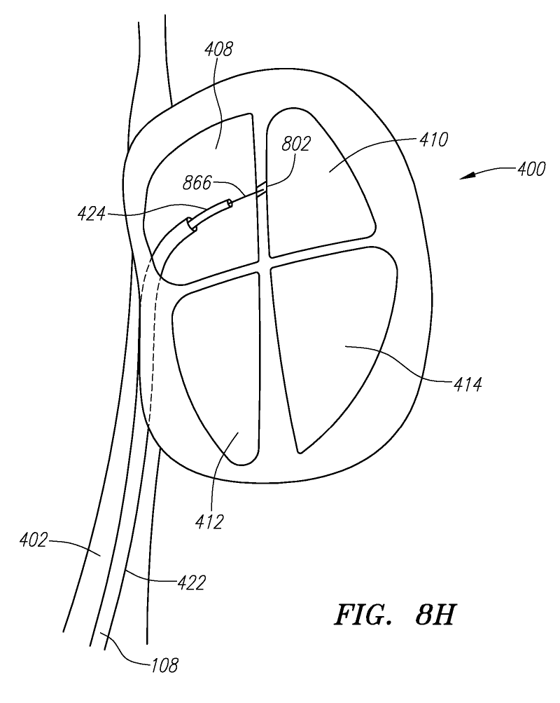

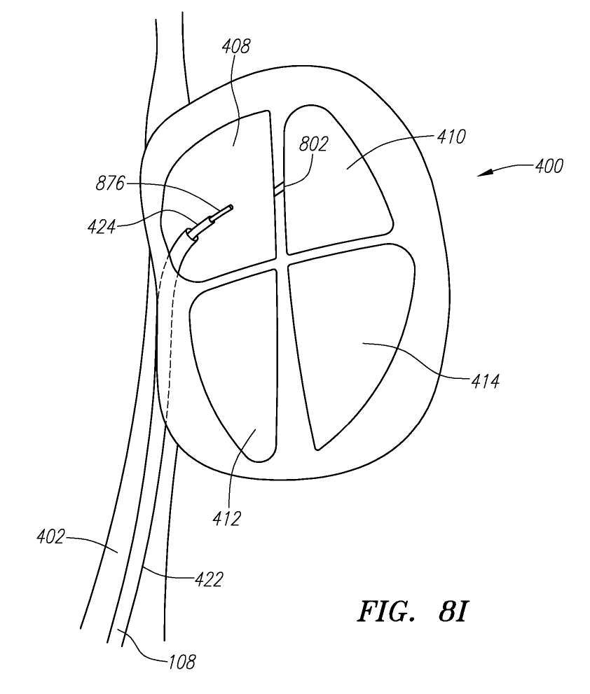

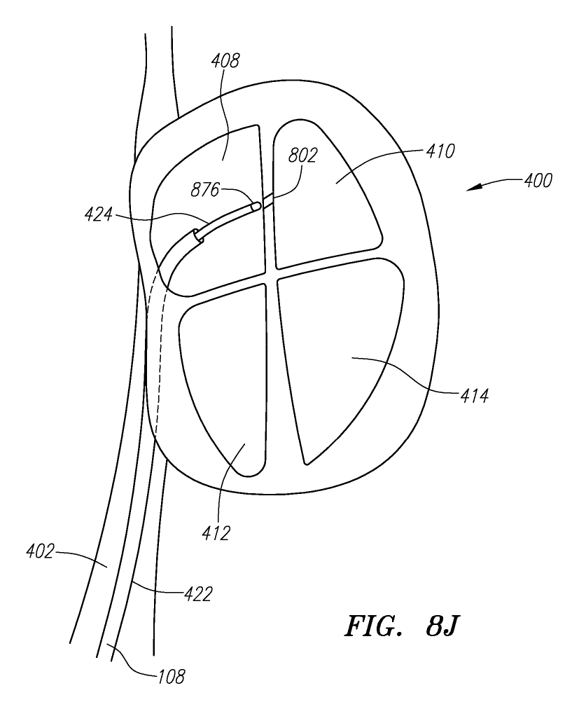

Referring to FIG. 8A, a sheath (422) and guide (424) instrument assembly (108) may be utilized to direct a laser fiber (826) to the location of a PFO (802) and use laser energy to ablate or “weld” the PFO (802) shut with a concomitant inflammation reaction. Referring to FIG. 8B, an ablation tool (836) is threaded through the working lumen of an instrument assembly (422, 424, 108) may be similarly used to tack a PFO (802) shut and induce a localized healing response. Referring to FIGS. 8C and 8D, a suturing tool (846) may be utilized to suture a PFO (802) shut. Referring to FIGS. 8E and 8F, a clip applying tool (856) may be utilized to clip a PFO (802) into a shut position. Referring to FIGS. 8G and 8H, a needle tool (866) advanced through the working lumen of a sheath (422) and guide (424) which are subsystems of the instrument assembly (108) may be utilized to irritate the tissue surrounding and/or forming the PFO (802), via full or partial thickness insertions of the needle (866) into the subject tissue, to induce a healing response sufficient to “scar” the PFO (802) shut. Referring to FIGS. 8I and 8J, an irritation tool (876) may be utilized to contact-irritate the subject tissue and induce a subsequent scarring shut of the PFO (802).

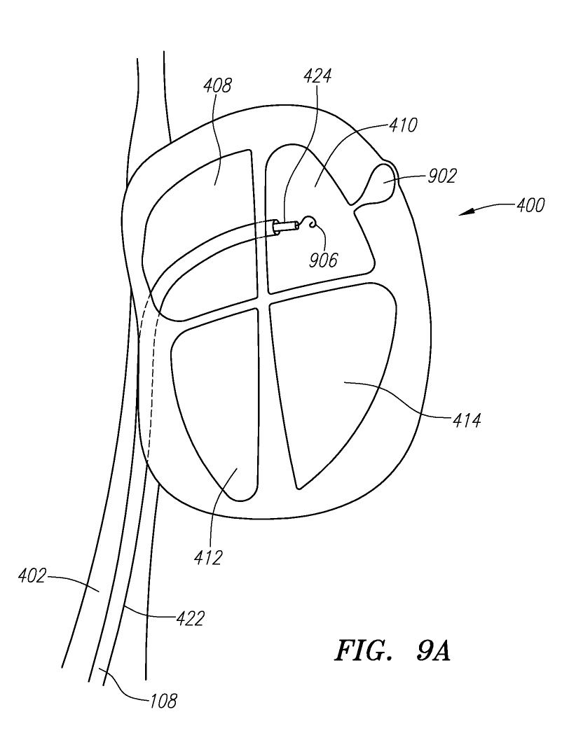

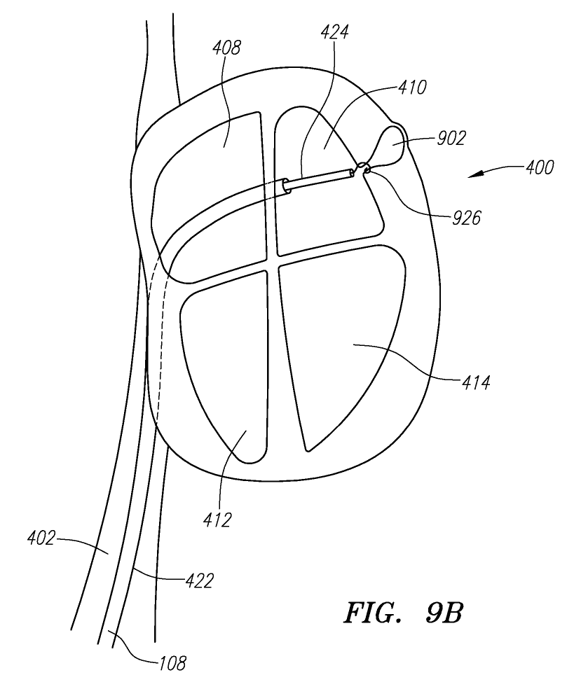

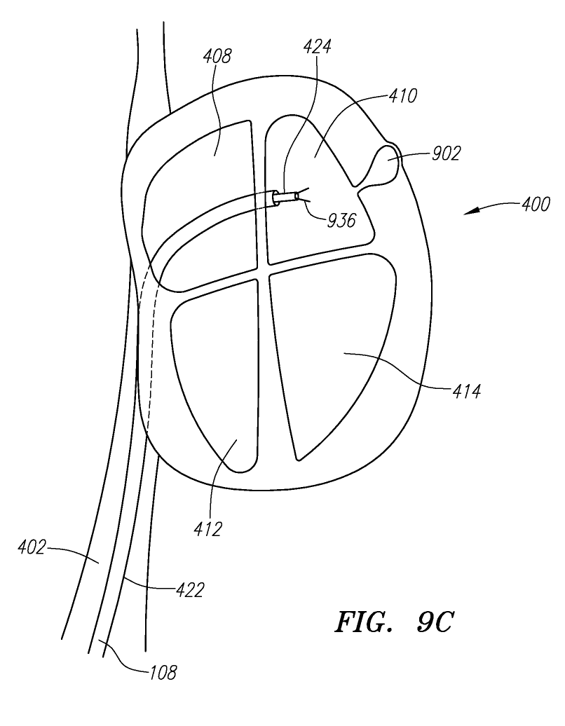

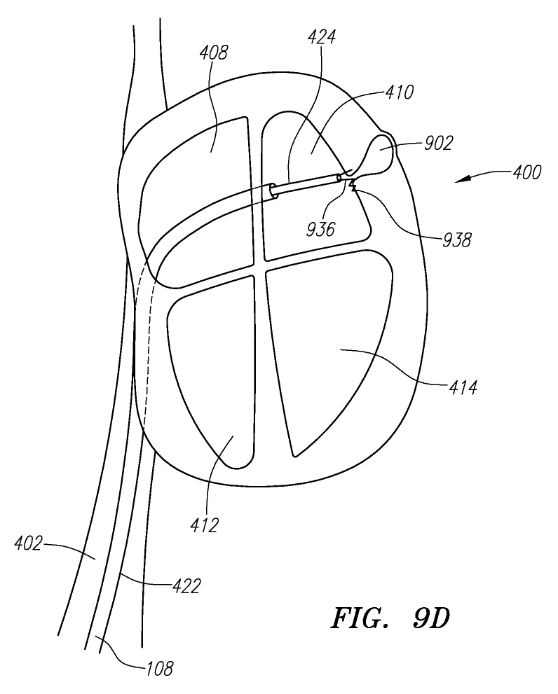

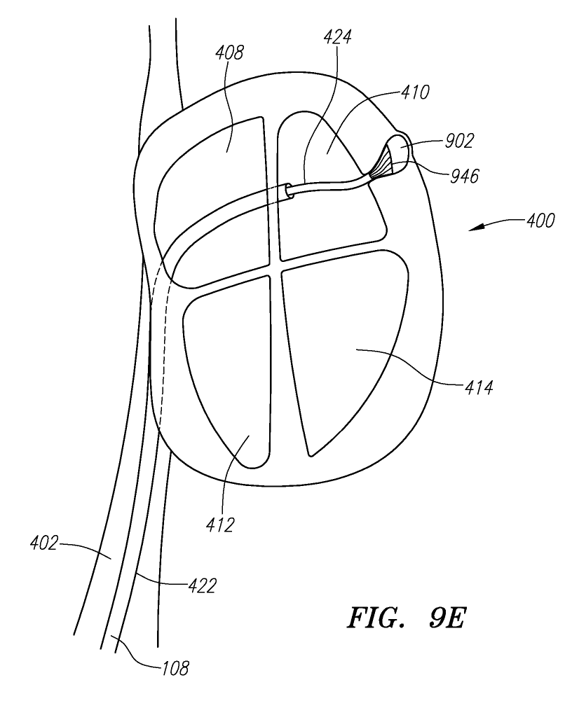

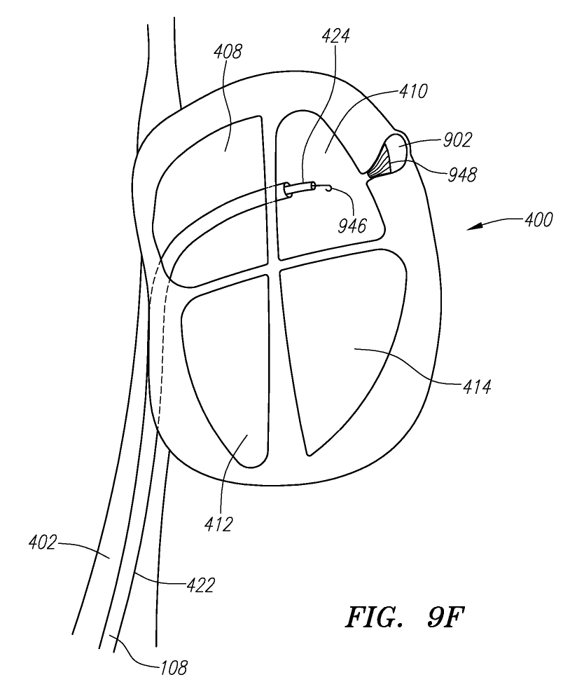

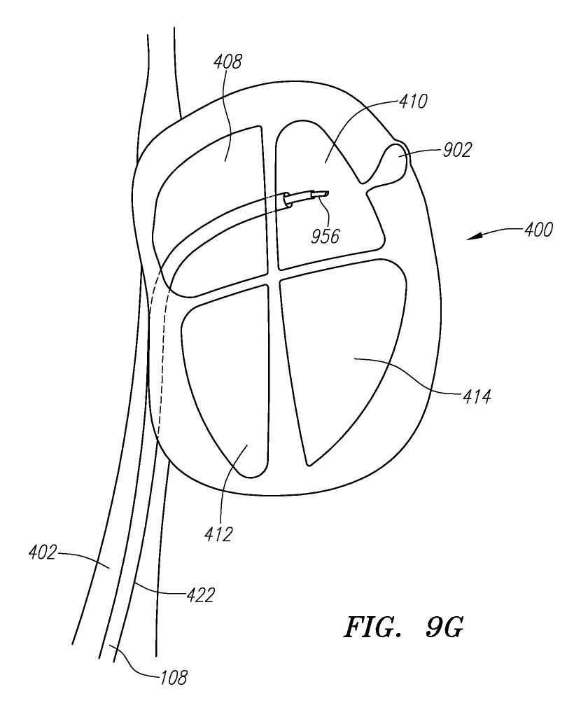

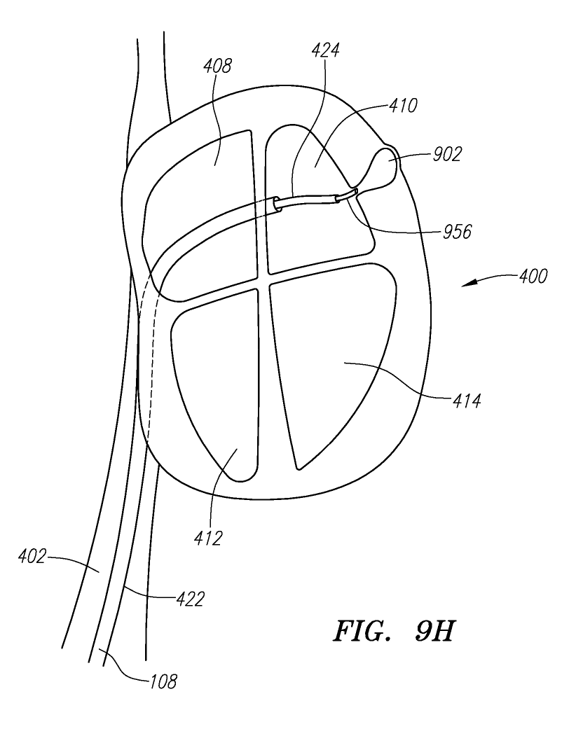

Left atrial appendage occlusion is anther cardiac abnormality. One of the significant clinical risks associated with atrial rhythm abnormalities is the development of blood clots in the atrial chamber which can result in stroke. An anatomic portion of the left atrium, referred to as the left atrial appendage (“LAA”) is particularly susceptible to clot formation. One approach to eliminate the risk of clot formation in the LAA is the use of catheter-based devices that are capable of blocking blood flow and pooling of blood in the LAA, thereby reducing the risk of forming blood clots in the atrium. These devices may work well if they could be properly positioned and oriented at the opening of the LAA. Such precise placement can be exceedingly challenging with conventional catheter techniques. Embodiments of the present invention facilitate the process of performing the aforementioned procedure and accurately navigating the devices necessary to address the LAA. Referring to FIGS. 9A and 9B, a suturing tool (926) may be utilized to close the entrance of an LAA, as facilitated by a robotic instrument assembly such as that depicted (108, 422, 424). Similarly, a clip application tool (936) applying a clip (938), expandable prosthetic tool (946) applying expandable prosthetic (948) (such as that available from Atri-Tech corporation under the trade name “Watchman”, and ablation tool (956) (i.e., to induce tissue welding to shut the entrance of the LAA) may be utilized to address the dangers of an open LAA, as depicted in FIGS. 9C-9H.

Pacing Lead Placement is another procedure performed to address cardiac abnormalities. Pacemakers have been used in cardiology for many years to treat rhythm abnormalities and improve cardiac function. More recently, many physicians have concluded that synchronistical pacing both ventricles of the heart is, in many patients, more effective than provide pacing at one ventricular location of the heart. This technique requires that one of the pacing leads be positioned at an optimal location in the wall of the left ventricle. In order to deliver the left ventricular lead, cardiologists often use a catheter based approach that delivers the pacing lead by introducing a cannula or tube into the coronary sinus. The coronary sinus is a vein that runs along the outside surface of the heart. Navigating this coronary sinus vein requires significant catheter manipulation and control. In addition, it also requires stability of the catheter tip when the proper anatomic location has been reached. Embodiments of the present invention facilitate placement of biventricular leads to their optimal locations to achieve the desired results.

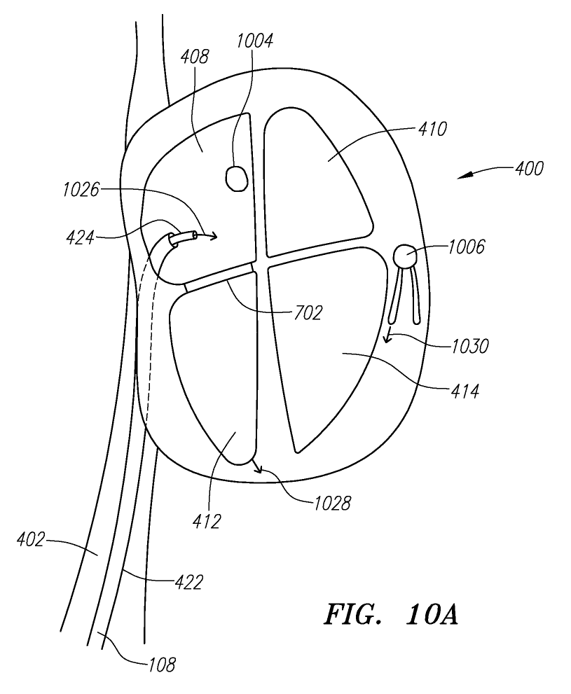

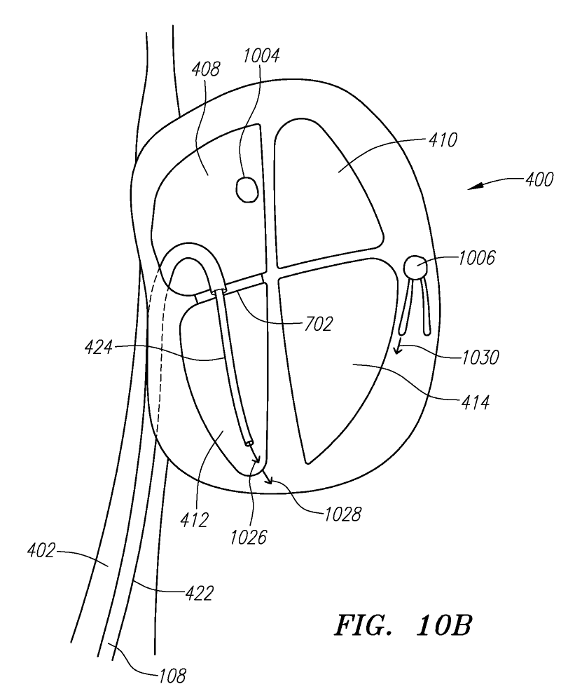

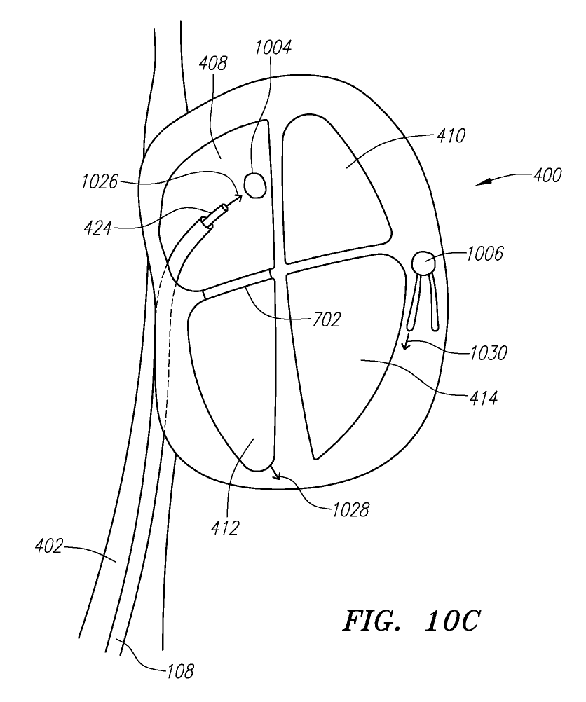

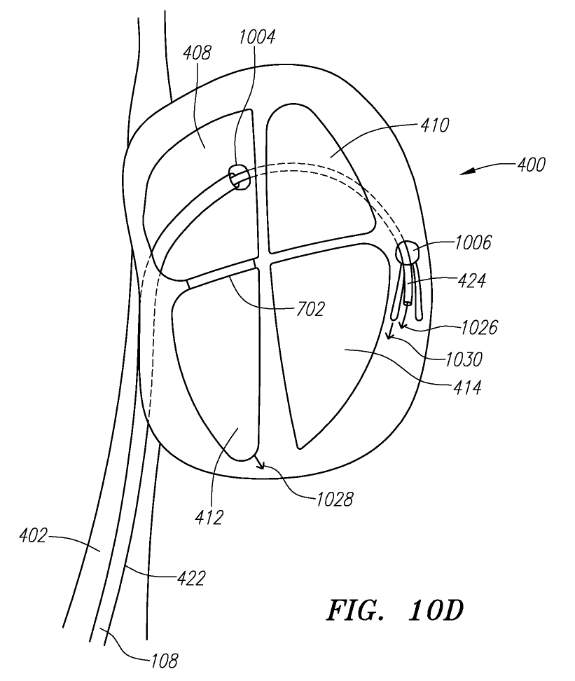

Referring to FIGS. 10A-10B, a sheath (422) and guide (424) instrument assembly (108) carrying a lead deploying tool (1026) may be advanced across the tricuspid valve (702) to press a lead (1028) into place at a targeted location (1002), such as a location adjacent the right ventricular apex. Referring to FIGS. 10C-10D, another pacing lead (1030) may be deployed at another targeted position by advancing a guide instrument (424) with a lead deploying tool (1026) through the coronary sinus (1004) to a desired location, such as a location adjacent or within one of the branches off of the coronary sinus in the left ventricular myocardium.

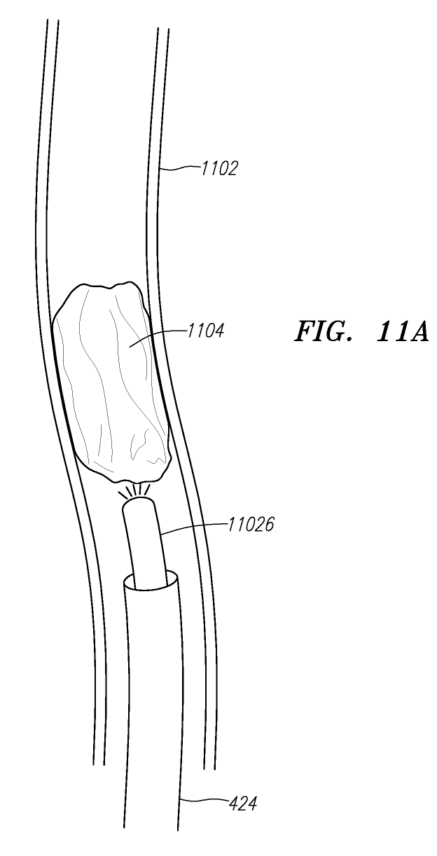

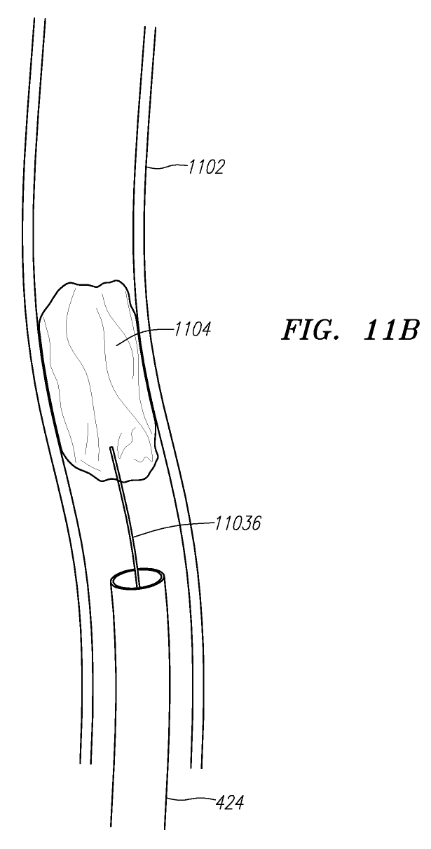

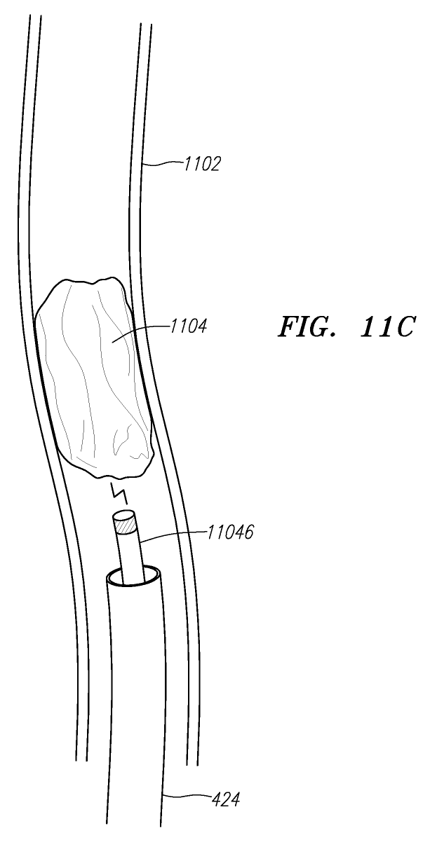

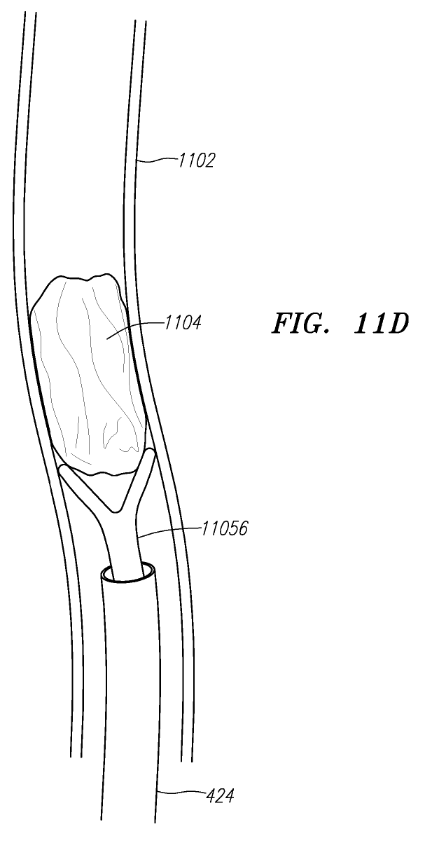

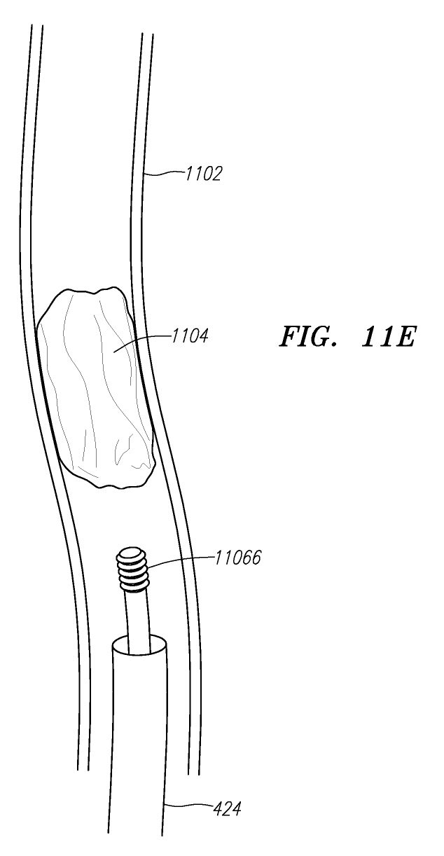

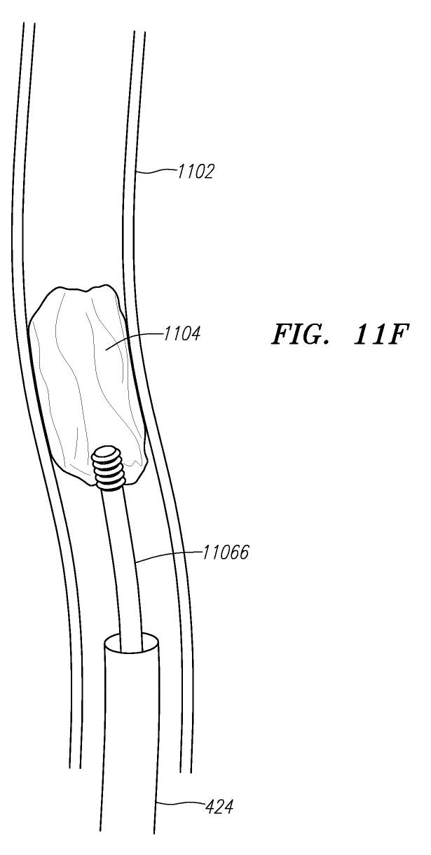

Chronic Total Occlusion (“CTO”) is another cardiac malady or condition that may be addressed by using the robotic surgical system (100). Chronic total occlusions generally are blockages of the coronary vasculature system which prevent blood from passing. These occlusions create inadequate blood flow to the region of the heart that derives its blood from the occluded artery, and forces the affected region to survive based on collateral circulation from other vessels. Unlike partial occlusions, CTOs are difficult to pass a catheter or guide wire through because of the lack of any central lumen in the artery. As a result, conventional therapy of balloon dilation and stent placement is often impossible to perform, and the atrial lesion may be left untreated. Many specialized devices have been developed to try to pass through the center of a CTO lesion. However, procedures using these devices are often lengthy and are associated with significant complications and unsuccessful outcomes due to calcification of the lesion or inability to navigate the catheter tip through the center of the artery. The subject robotic catheter system (100), because of its ability to precisely control and stabilize the tip of the catheter as it is advanced, facilitates the crossing and removal of CTOs. For example, referring to FIG. 11A, a sheath (422) and guide (424) instrument assembly (108) may be utilized to advance an RF ablation tool (11026) into position where a CTO (1104) may be ablated with precision and destroyed and/or removed in a coronary artery (1102). FIG. 11B depicts another embodiment wherein an RF guidewire (11036) is advanced to destroy and/or remove a CTO (1104) in a coronary artery (1102). FIG. 11C depicts another embodiment wherein a laser fiber (11046) is utilized to destroy and/or remove a CTO (1104). FIG. 11D depicts another embodiment wherein a very small grasping tool (11056) is utilized to destroy and/or remove a CTO (1104). FIGS. 11E-11F depict another embodiment wherein a cutting/removing tool (11066), such as those available from Fox Hollow Corporation is utilized to destroy and/or remove a CTO (1104).

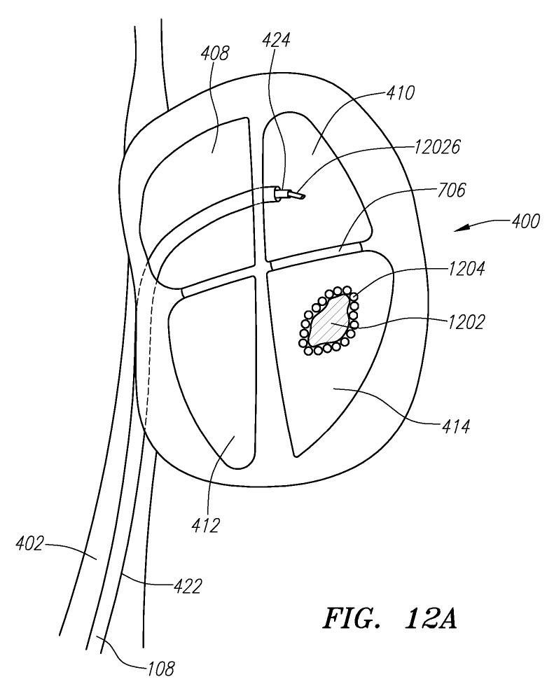

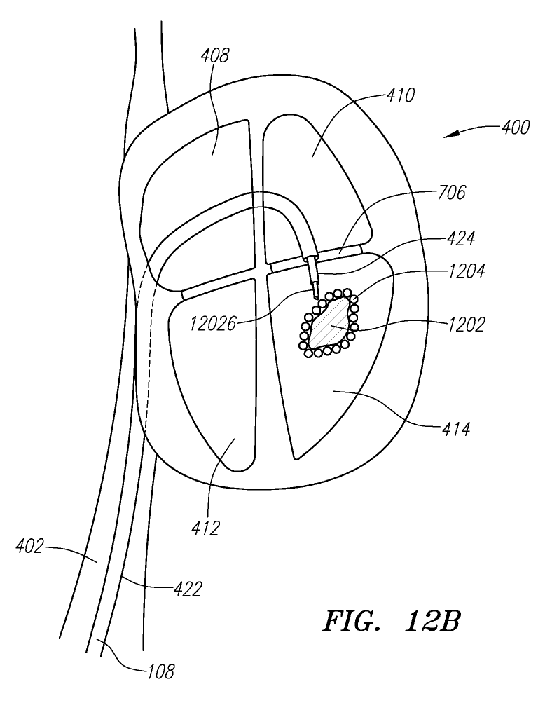

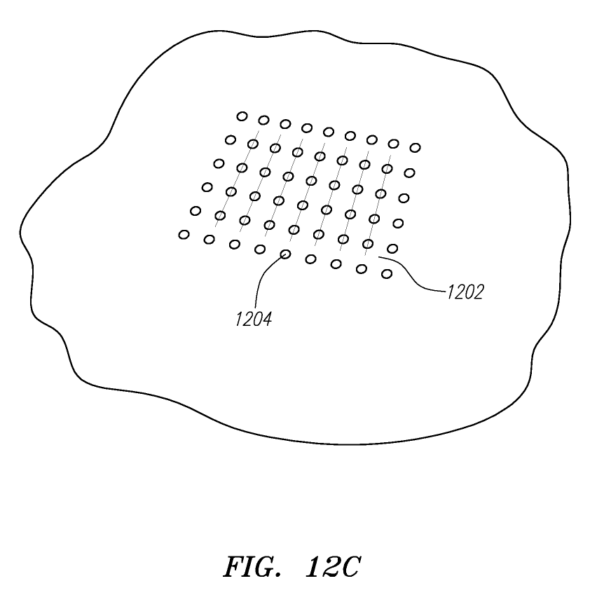

Robotic surgical system (100) may also be used to perform ventricular injection therapy. Many chronic heart maladies cause progressive deterioration of heart functions that often resulting in debilitating and fatal conditions commonly referred as congestive heart failure (“CHF”). In CHF, the heart muscle becomes less efficient, the chambers of the heart begin to dilate and cardiac function tends to deteriorate. As the heart muscle becomes weaker, the heart has to work harder to pump adequate amount of blood through the circulatory system. The harder the heart has to work, the more damage may be done to its structure and function. Typically, clinicians treat CHF with a variety of drugs that substantially decrease blood volume and increase contractility of the heart muscle. Recently, there have been investigations of techniques that could repair damaged muscle cells by directly injecting growth factors or healthy cells into injured or damaged muscles. These techniques have shown some promising results of healing the damaged muscle; however, these techniques require the drugs to be applied directly to the damaged muscle. Accordingly, the needle injector for delivering the drug to the damaged muscle in the heart must be precisely and accurate controlled in order to ensure direct delivery of the drugs to the damaged muscle. The subject robotic surgical system (100) is an effective means for delivering ventricular injections at the precise locations where clinicians desire to deliver drugs and cell therapies. Referring to FIGS. 12A-912B, an injection tool (12026) may be operatively coupled to the sheath (422) and guide (424) instrument assembly (108). The assembly (108, 422, 424, and 12026) is advanced trans-septally into the left atrium, across the mitral valve, and into the left ventricle (414), as illustrated in the figures. With the guide instrument (424) advanced into the left ventricle (414) along with the injection tool (12026), a precision pattern (1204) of injections may be made, for example, around an infracted tissue portion (1202), to start revascularization and/or rebuilding of such portion. In one embodiment, the pattern (1204) may be in a pattern of a matrix as illustrated in FIG. 12C. Several subsequent treatments may be applied to increase the rebuilding of such portion of tissue.

The robotic surgical system (100) may be used to perform a valve repair procedure. Heart valve disease is a common disorder which affects millions of patients and is characterized by a progressive deterioration of one or more of the heart's valvular mechanisms. Repair of heart valves has historically been accomplished by open heart surgery. Although such open heart surgery is often successful in improving valve function, however, there is also a high risk of death associated with open heart or heart valve surgeries. Even if such surgery is successful, there is a long period of post-operative recovery associated with open heart surgery. As a result, cardiologists tend to wait as long as possible before resorting to surgery in patients with deteriorating valve function.

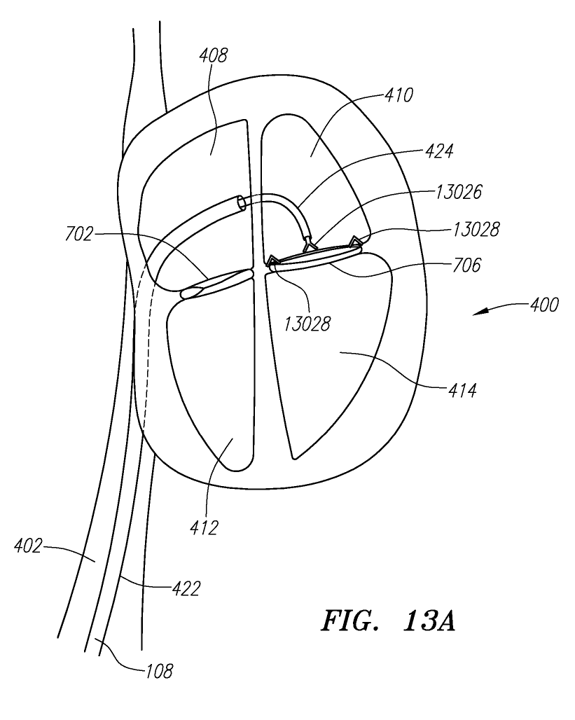

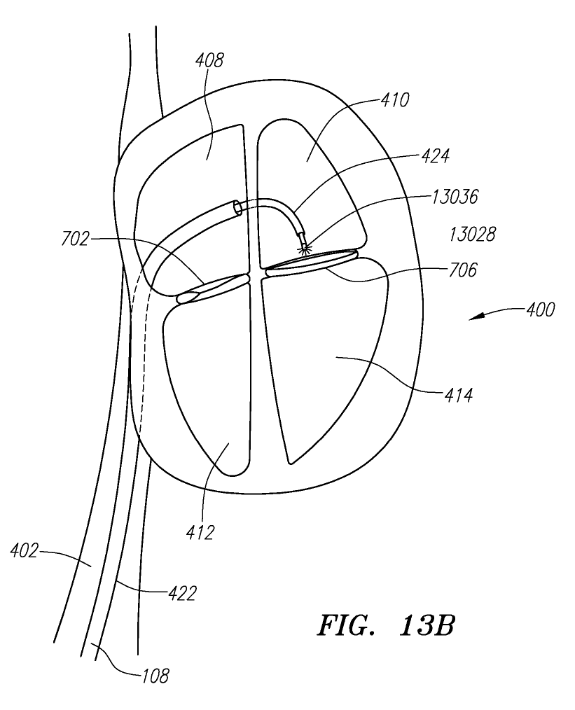

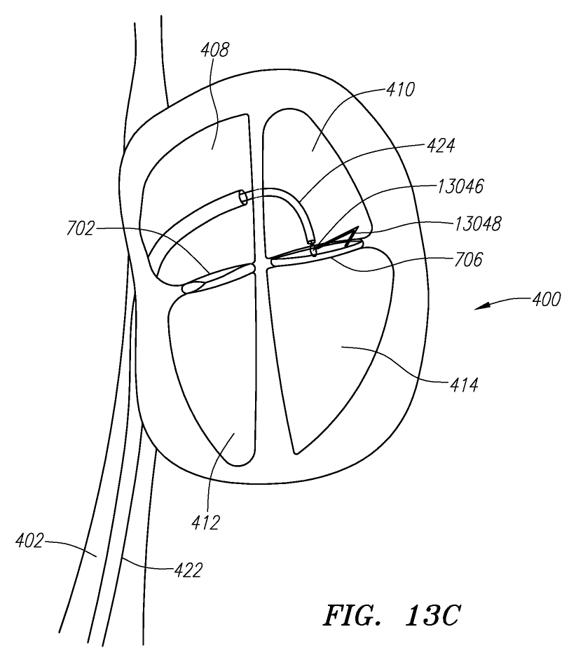

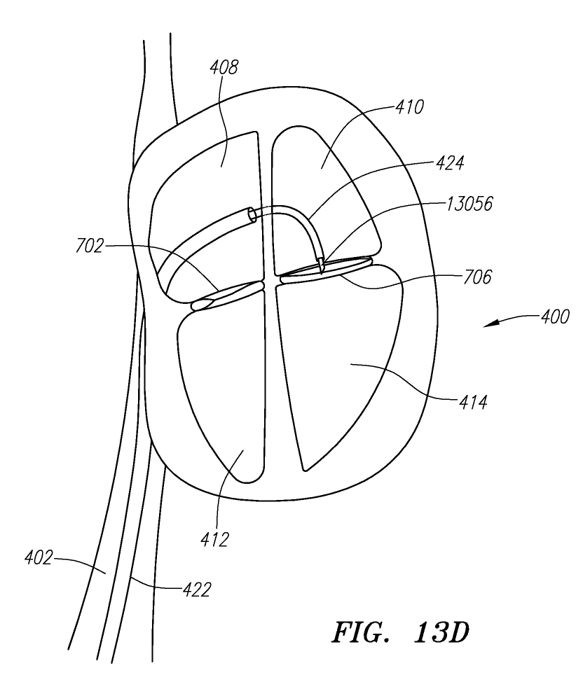

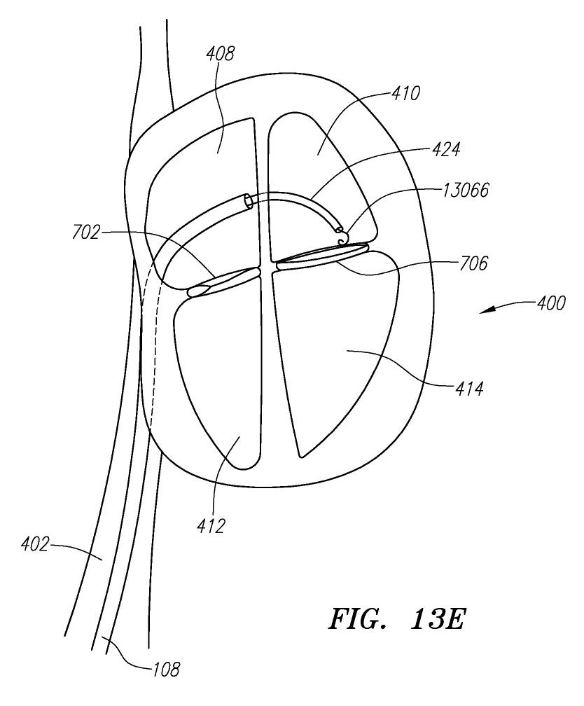

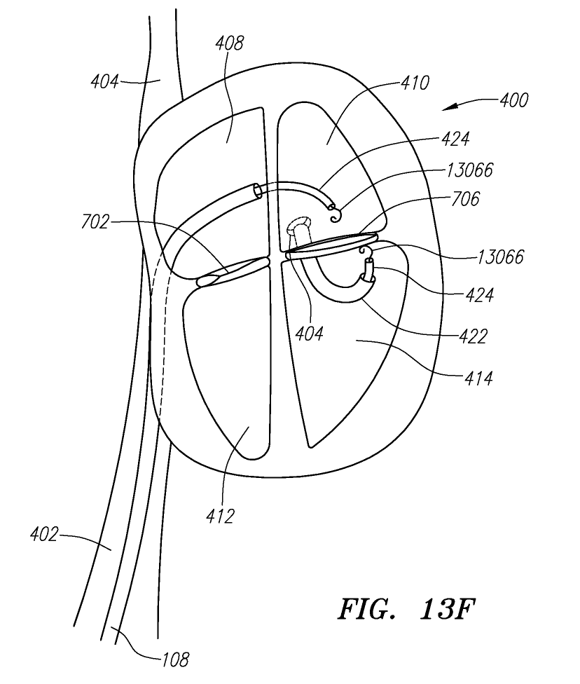

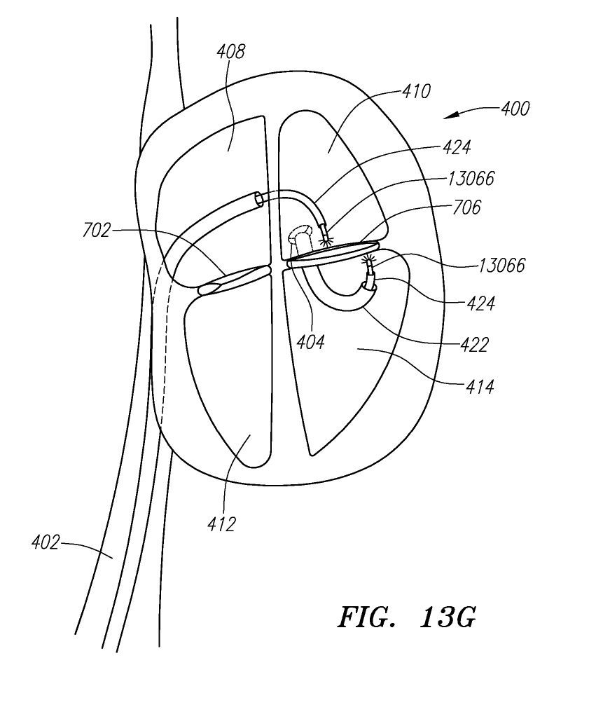

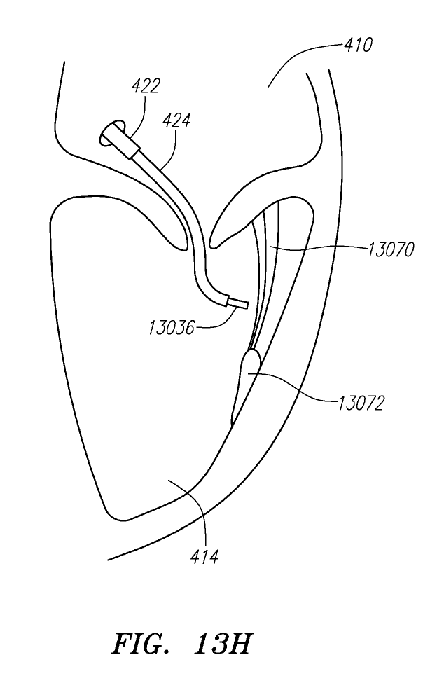

There is increasing interest in treating valve disease with less invasive procedures in order to encourage treatment in the earlier stages of the disease and potentially slow or stop the progression of heart failure. In recent years, catheter-based procedures have been developed for repairing valves in a surgical manner. As these procedures develop, physicians require a new generation of catheters that can be used like surgical tools and which can be precisely controlled, as may be provide by the subject robotic catheter system (100). Referring to FIG. 13A, a clip deployer (13026) may be utilized to deploy clips (13028) around the mitral annulus and adjust the geometry of the annulus. FIG. 13B depicts an ablation tool (13036) utilized to induce localized ablations to adjust or shrink the geometry of the mitral annulus. Similarly, an ablation tool (13036) may be used to adjust or shrink the geometry of the mitral valve leaflets. FIG. 13C depicts a clip or suture deploying tool (13046), such as those available from E-Valve Corporation, to position a clip or suture (13048) across the mitral leaflets in an Alfieri technique procedure, utilizing the precision and stability of the sheath (422) and guide (424) of the instrument assembly (108). FIG. 13D depicts a sheath (422) and guide (424) of instrument assembly (108) delivering a resecting tool (13056) which may be utilized to resect the mitral leaflets and improve coaptation. FIG. 13E depicts an antegrade approach using a suture tool (13066) to deploy sutures into the mitral annulus to modify the geometry of the mitral valve. FIG. 13F depicts both antegrade and retrograde instrument assemblies (e.g., 13066, etc.) to deploy sutures into the mitral annulus. FIG. 13G depicts both antegrade and retrograde ablation of the mitral annulus, for example by a bipolar electrode configuration formed by the electrodes carried by the opposing instrument assemblies (e.g., 13066). FIGS. 13H and 13I illustrate the positions of the mitral valve leaflets may be adjusted by adjusting (e.g., shortening, etc.) the length of the leaflet chords (13070), chordae tendineae (13070), or papillary muscle (13072) to ensure proper closure and/or alignment of the leaflets to prevent leakage by using a clip tool (13026) to deploy a clip (13028), an ablation tool (13036), a suturing tool (13046), etc.

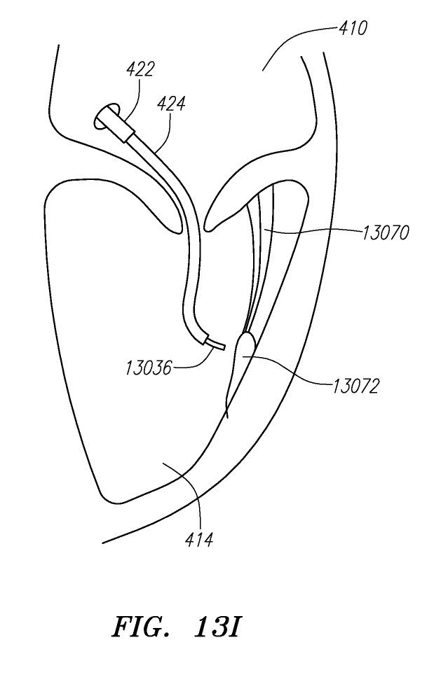

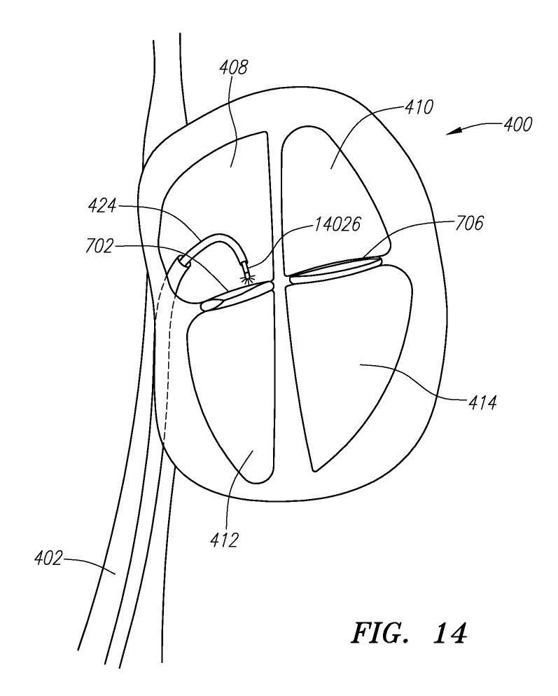

FIG. 14 depicts an ablation tool (14026), similar to the description and procedure as described above, modifying the geometry of the tricuspid valve (702). The configurations of tools similar to those as illustrated in FIGS. 13A-13G may be utilized on the tricuspid valve (702).

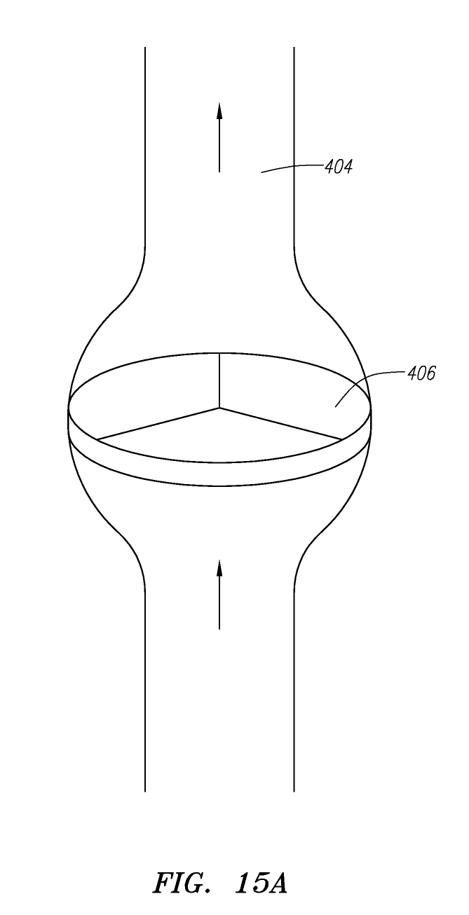

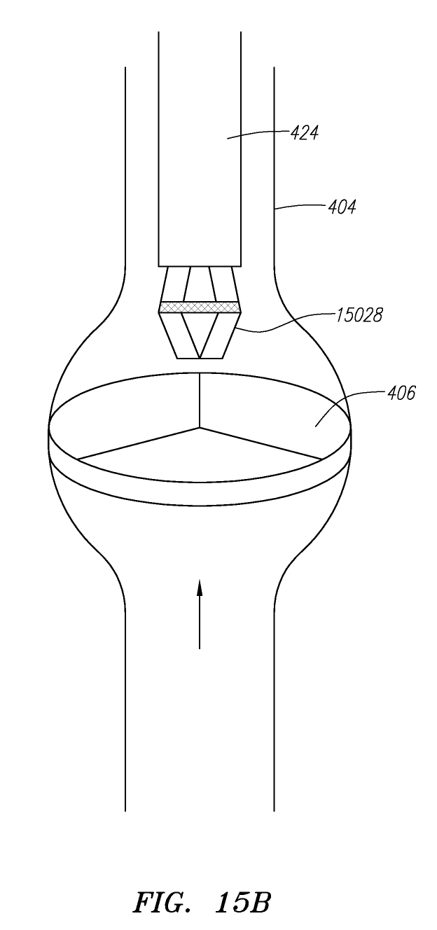

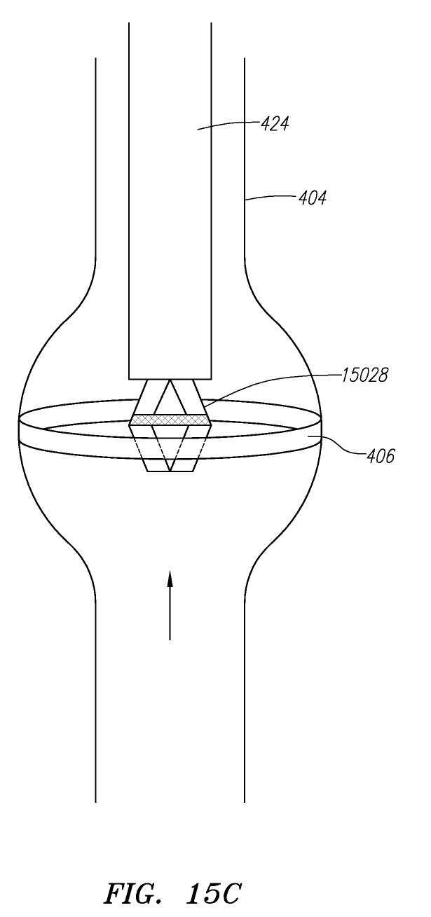

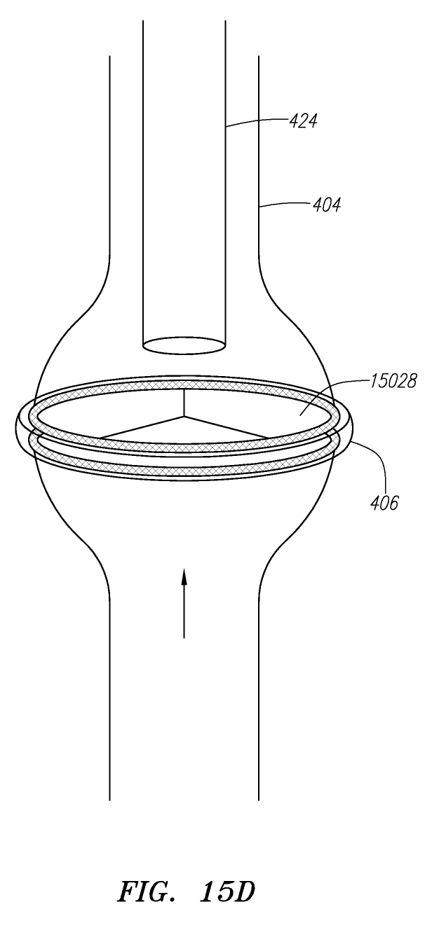

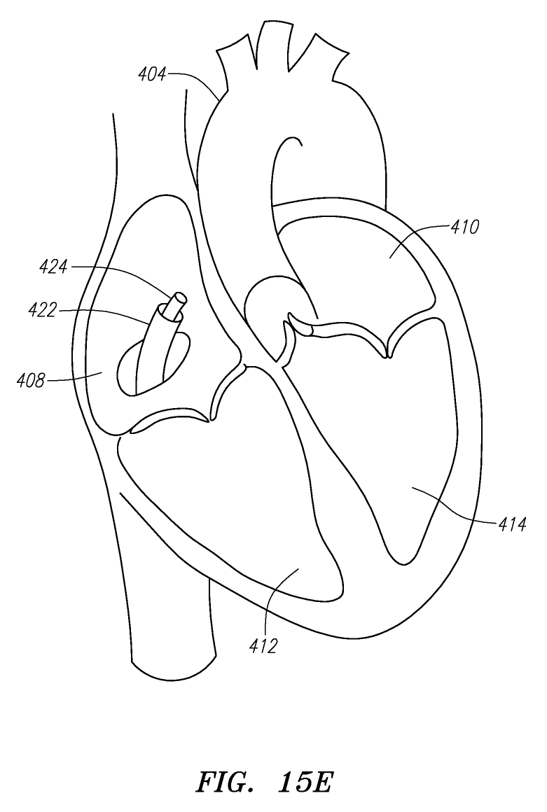

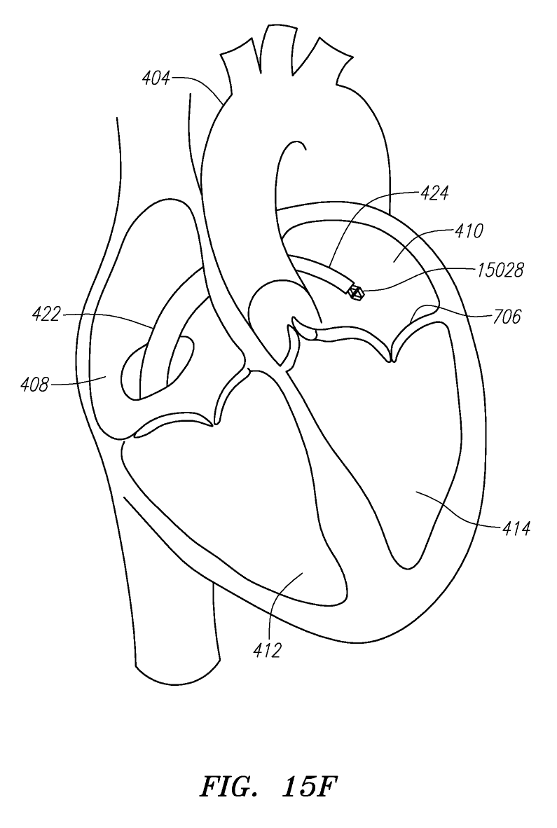

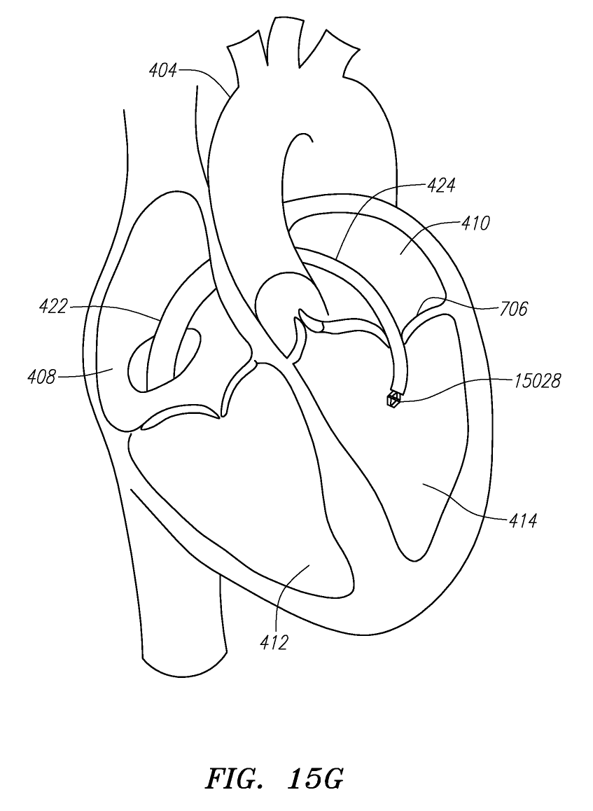

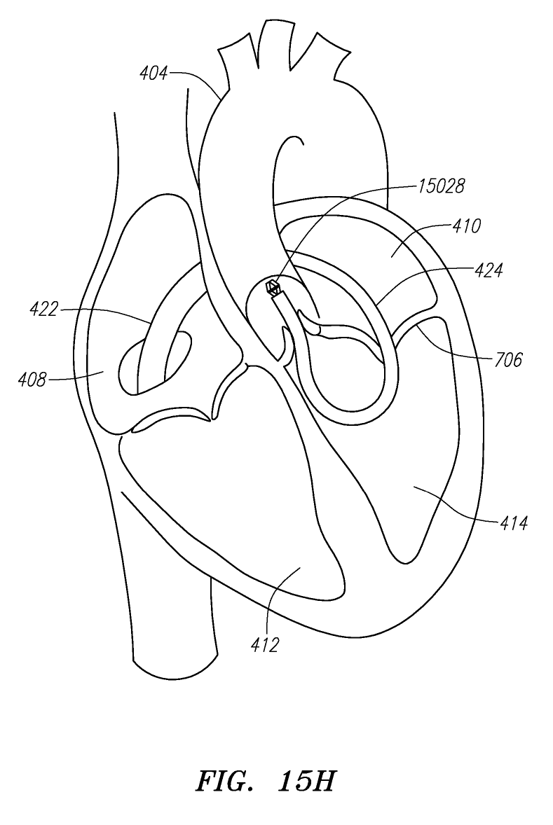

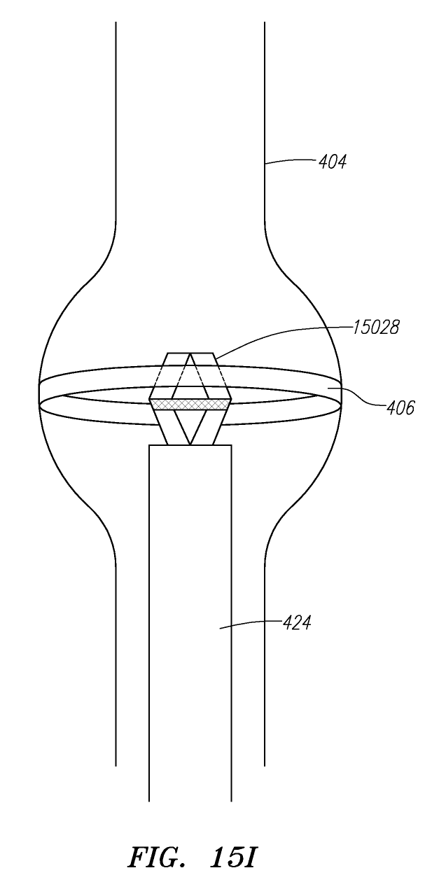

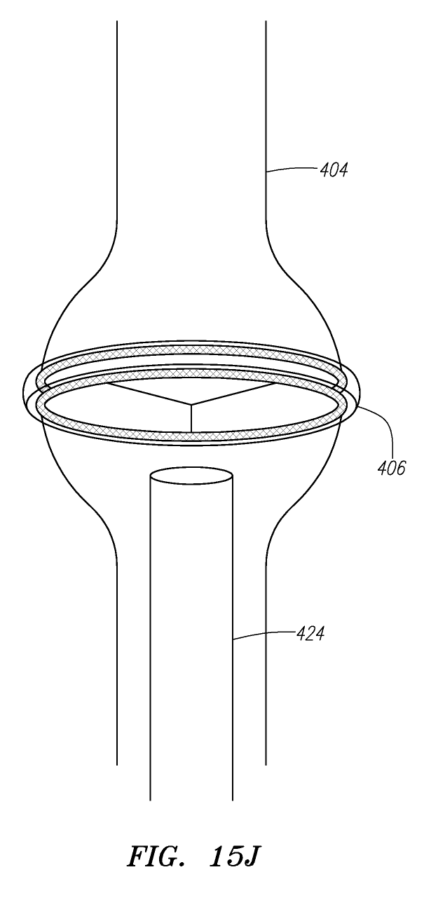

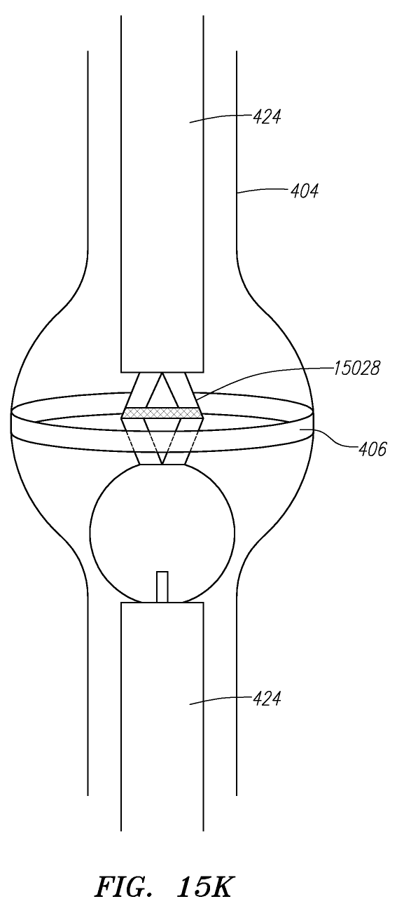

FIG. 15A through FIG. 5D depict a robotic instrument assembly (108) using a retrograde approach to deploy an expandable aortic valve prosthetic (15028). Alternatively, FIG. 15E through FIG. 15J illustrate a robotic instrument assembly (108) being used by way of the inferior vena cava through the septum and the mitral valve, and then going up the aorta to deploy an expandable aortic valve prosthetic (15028) in the aorta. The methods as described may be referred a “single-handed” approach. That is, the expandable aortic valve prosthetic (15028) may be deployed by the method as illustrated in FIGS. 15A through 15D or the method as illustrated in FIG. 15E through FIG. 15J using one instrument assembly (108). Alternatively, the expandable aortic valve prosthetic (15028) may be deployed using a “two-handed” approach. That is, the expandable aortic valve prosthetic may be deployed using two robotic instrument assemblies (108). For example, a first instrument assembly (108) may be used to position or adjust the placement of the aortic valve prosthetic (15028) while a second instrument assembly (108) may be used to place the aortic valve prosthetic. FIG. 15K, illustrates one embodiment of a two-handed approach. As illustrated in FIG. 15K, an expandable valve prosthetic (15028) is being deployed by a first instrument assembly (108-422, 424) using a retrograde approach as illustrated in FIG. 15A through FIG. 15D. At the same time, a second instrument assembly (108-422, 424) with a positioning apparatus (e.g., a balloon with a scope, etc.) approaches the aortic valve (406) from different direction of deployment for the valve prosthetic (15028), such that the positioning apparatus assists with the placement or positioning of the prosthetic (15028) as it is being deployed.

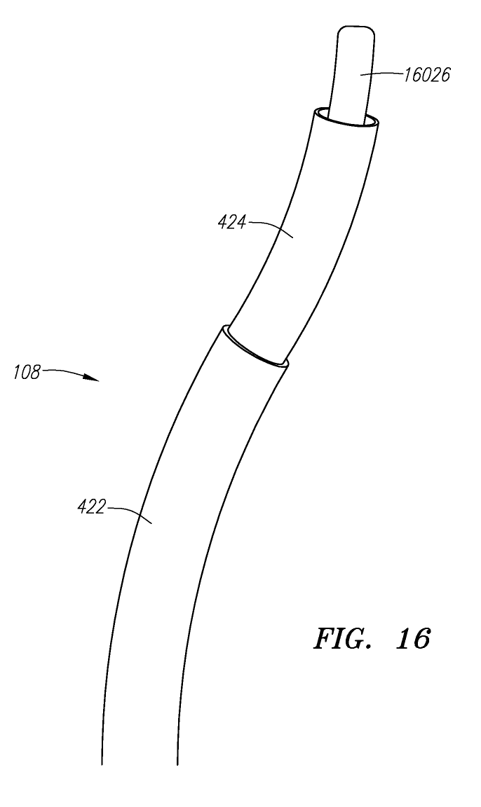

In addition, the robotic surgical system (100) including the control station (102), instrument driver (106), instrument (108), and the wired connection (112) may be used to treat other diseases, maladies, or conditions in the tissues or organs of the digestive system, colon, urinary system, reproductive system, etc. For example, the robotic surgical system (100) may be used to perform Extracorporeal Shock Wave Lithotripsy (ESWL). FIG. 16 illustrates one embodiment of instrument (108) configured to perform ESWL. As illustrated in FIG. 16, instrument (108) may include a sheath catheter (422), a guide catheter (424), and a lithotripsy laser fiber (16026). Analogous to the discussion above, components or subsystems of the instrument (108) may be guided, manipulated, or navigated to the kidney to perform various operations. For example, subsystems of the instrument (108) may be guided, manipulated, or navigated to the kidney to remove kidney stones as oppose to similar components or subsystems of embodiments of the instrument (108), e.g., an ablation catheter, being guided, manipulated, or navigated to the left atrium of the heart to performing cardiac ablation to address cardiac arrhythmias. The lithotripsy laser fiber (16026) may include a quartz fiber coupled, connected to, or associated with a laser, such as a Holmium YAG laser, to apply energy to objects such as kidney stones, etc. In one configuration, the laser source may be positioned and interfaced with the fiber (16026) proximally, as in a typical lithotripsy configuration, with the exception that in the subject embodiment, the fiber (1602) is positioned down the working lumen of one or more robotic catheters (e.g., sheath catheter (422) and guide catheter (424)). All the necessary power source and control mechanisms including hardware and software to operate the laser may be located in the electronics rack (114) near the operator control station (102) of the robotic surgical system (100)

Since the distal tip of the lithotripsy fiber (16026) is configured to deliver energy to a target object, such as a kidney stone, the distal tip may be more generically described as an energy source. Indeed, in other embodiments, other energy sources, besides a laser, may be used to affect tissue. For example, in other embodiments, the energy source may be comprised of an RF electrode, an ultrasonic transducer, such as a high-frequency ultrasonic transducer, or other radiative, conductive, ablative, or convective energy source.

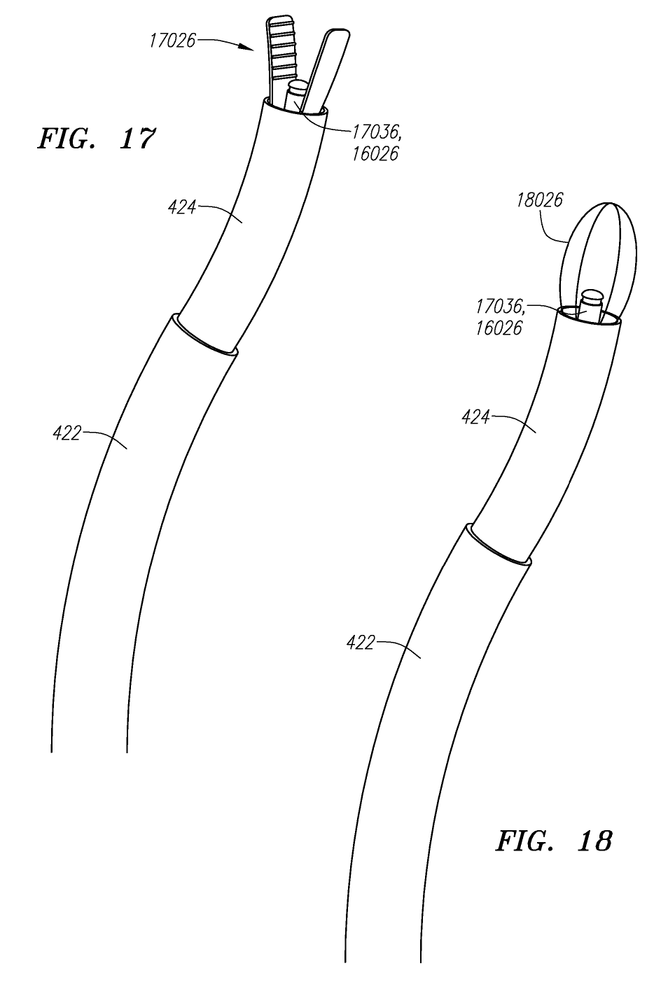

As may appreciated, the components or subsystems of instrument (108) may be configured with numerous different instruments or tool for performing various minimally invasive operations. For example, FIG. 17 depicts a guide instrument (424) operatively coupled to a grasper (17026) fitted with an energy source (17036), such as a lithotripsy laser fiber (16026) in a configuration wherein an object, such as a kidney stone, grasped within the clutches of the grasper (17026), may also be ablated, destroyed, fragmented, etc, by applied energy from the source (17036), which is positioned to terminate approximately at the apex of the grasper (17026) which it is likely to be adjacent to captured objects.

FIG. 18 depicts a similar configuration as the instrument assembly (108) including the sheath (422) and guide (424) that is illustrated in FIG. 17. FIG. 18 illustrates a basket tool (18026) and energy source (17036), such as a lithotripsy fiber (16026), positioned through the working lumen of the guide instrument (424). In each of the configurations depicted in FIG. 17 and FIG. 18, the energy source (17036) may be coupled to the pertinent capture device, or may be independently positioned through the working lumen of the guide instrument (424) to the desired location adjacent the capture device (17026, 18026). Each of the tools described herein, such as graspers, baskets, and energy sources, may be controlled proximally as they exit the proximal end of the working lumen defined by the guide instrument (424), or they may be actuated manually, automatically or electromechanically, for example through the use of electric motors and/or mechanical advantage devices. For example, in one embodiment, a configuration such as that depicted in FIG. 18, the sheath (422) and guide (424) instruments are preferably electromechanically operated utilizing an instrument driver (106) (not shown in these two figures) such as that described in the aforementioned patent application Ser. No. (11/481,433). The grasping mechanisms (17026, 18026) may be manually actuated, for example utilizing a positioning rod and tension wire, or electromechanically operated using a servomechanism or other proximal actuation devices. The energy source (17036) may be operated proximally utilizing a switch, such as a foot pedal or console switch, which is associated with the proximal energy control device (not shown in FIGS. 17 and 18).

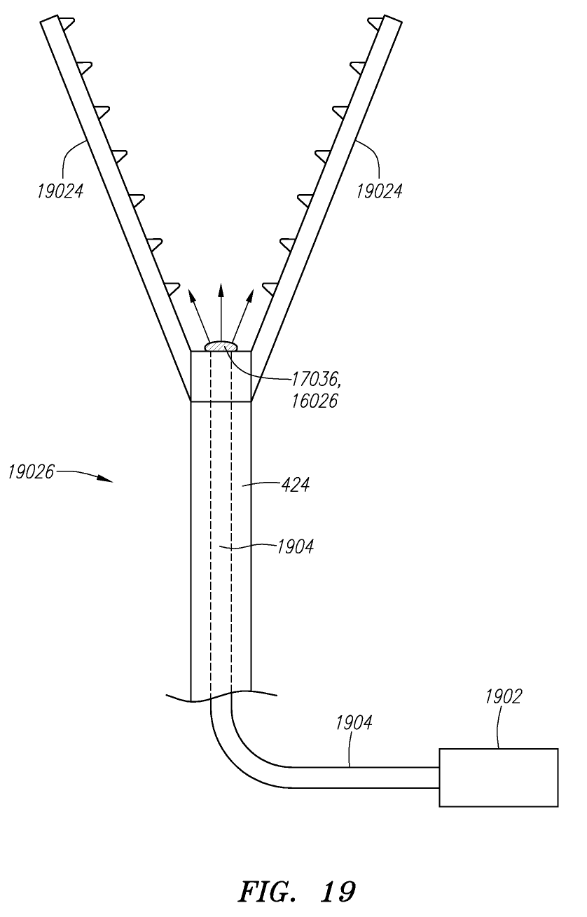

FIG. 19 depicts an expandable grasping tool assembly (19026) with an energy source (17036, 16026) mounted at the apex of the grasper mechanism. The energy source (17036, 16026) is proximally associated, by one or more transmission leads (1904), such as a fiber or wire, with a device (1902) such as an RF generator or laser energy source. The opposing jaws (19024) of the depicted grasping tool assembly (19026) are biased to spring outward, thus opening the grasper when unbiased. When pulled proximally into a confining structure, such as a lumen of a guide instrument (424), the hoop stress applied by the confining structure urges the jaws (19024) together, creating a powerful grasping action.

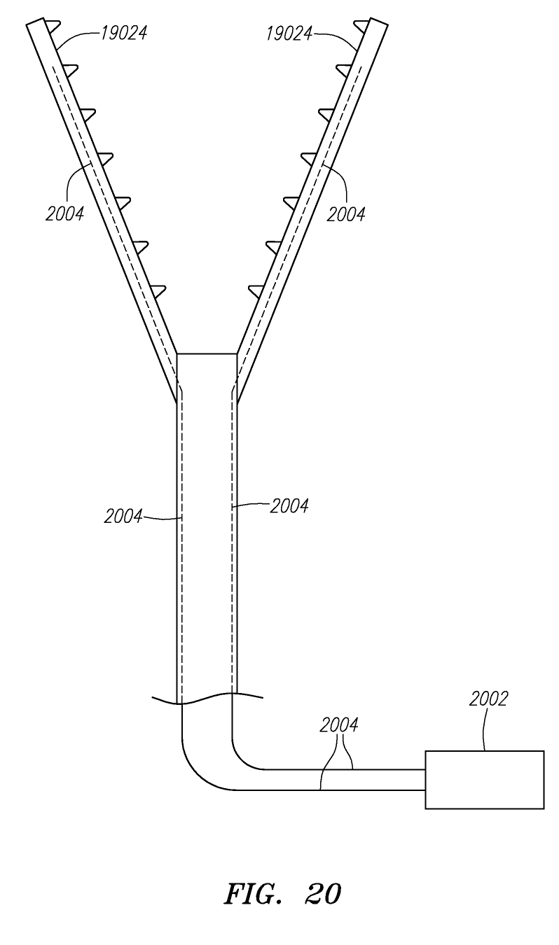

FIG. 20 depicts a bipolar electrode grasper with a proximally associated RF generator or other energy source (2002). In this embodiment, each of the jaws (19024) is biased to swing outward, as in the embodiment depicted in FIG. 19, and each of the jaws (19024) also serves as an electrode for the bipolar pairing, to be able to apply energy to items or objects which may be grasped. Leads (2004) are depicted to couple the jaws (19024) with a proximally positioned energy source (2002), such as an RF generator

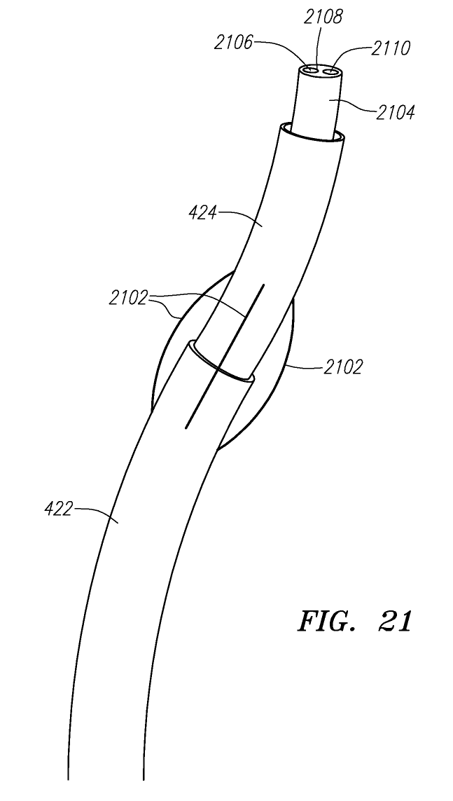

FIG. 21 depicts a sheath instrument (422) coupled to a group of basket arms (2102) that are biased to bend inward (i.e., toward the longitudinal axis of the sheath/guide as depicted), and configured to grasp a stone or other object as the guide instrument (424) is withdrawn proximally into the sheath instrument (422). The depicted embodiment features an image capture device (2104) which may or may not have a lens (2106), illumination fibers (2108) to radiate light, infrared radiation, or other radiation, and a working lumen (2110) for positioning tools distally. The image capture device (2104), which may comprise a fiberscope, CCD chip, infrared imaging device, such as those available from CardioOptics Incorporated, ultrasound device, or other image capture device, may be used, for example, to search for objects such as stones, and when located, the guide instrument (424) may be withdrawn into the sheath instrument (422) to capture the object, which the entire assembly is gently advanced to ensure that the object remains close to the distal tip of the assembly for easy capture by the basket device (2102).

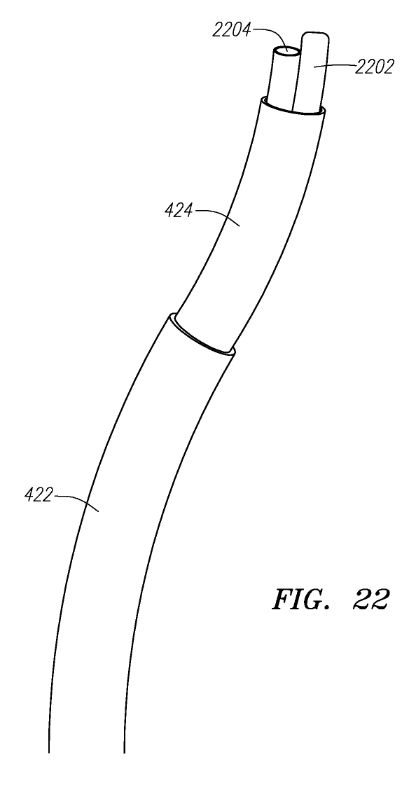

FIG. 22 depicts an assembly comprising a lithotripsy fiber (2202) and image capture device (2204) configured to enable the operator to see and direct the laser fiber (2202) to targeted structures, utilizing, for example, the high-precision navigability of the subject sheath (422) and guide (424) instrument assembly (108), and apply energy such as laser energy to destroy or break up such structures. Preferably the image capture device (2204) is positioned to include the position at which the energy source (such as a lithotripsy fiber 2202) as part of the field of view of the image capture device (2204)—i.e., to ensure that the operator can utilized the field of view to attempt to bring the energy source into contact with the desired structures.

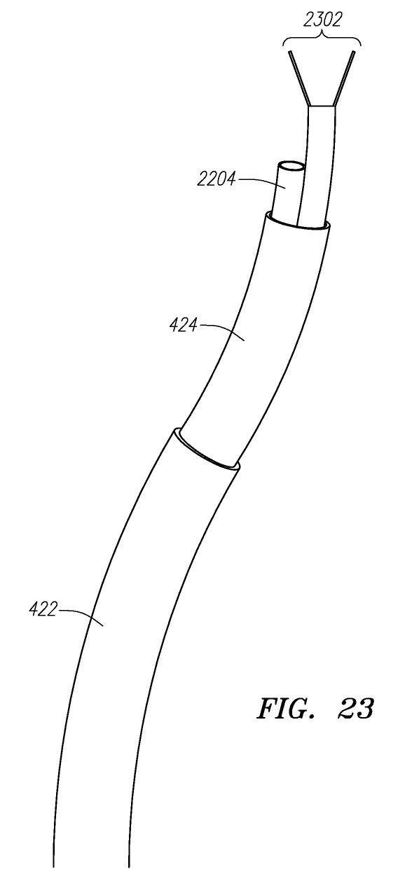

FIG. 23 depicts a similar embodiment as the one shown in FIG. 22, which includes a grasping tool (2302) to grasp a stone or other object and bring it proximally toward the image capture device (2204), such that it may be examined, removed proximally through the working lumen of the guide instrument (424), etc.

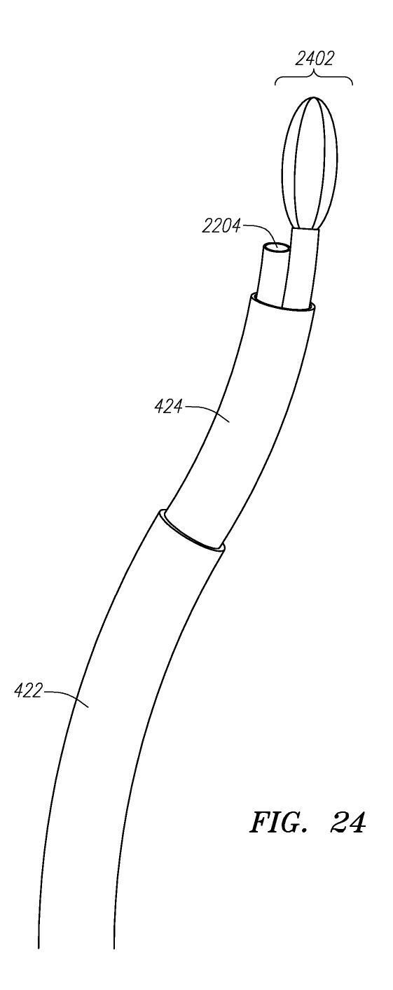

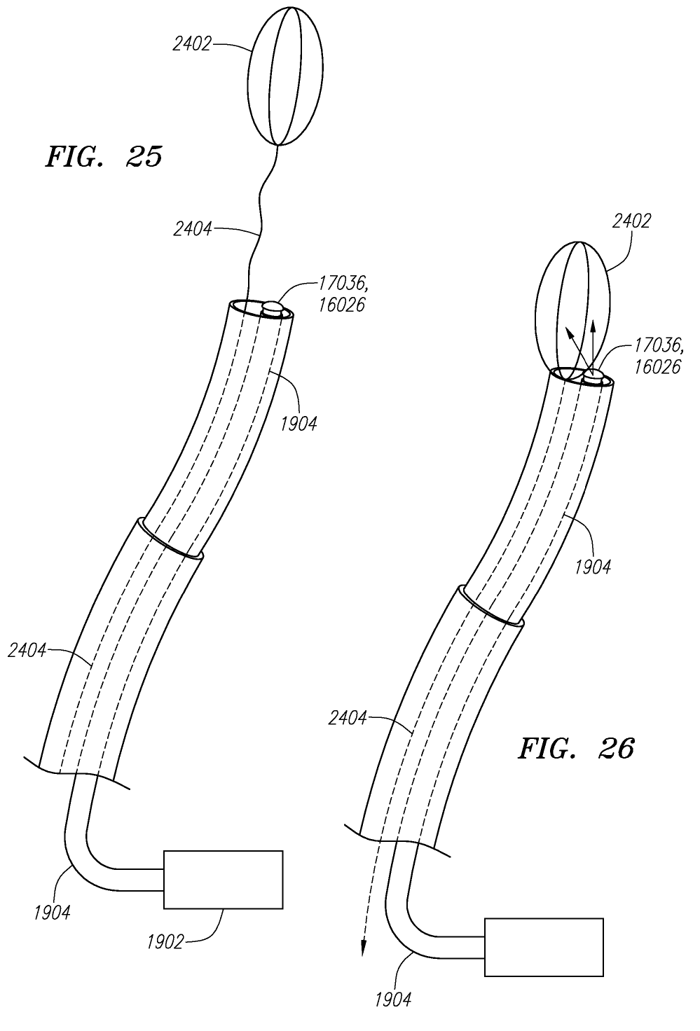

FIG. 24 illustrates another similar embodiment, which includes a basket tool (2402). FIG. 25 and FIG. 26, illustrate how an embodiment such as one depicted in FIG. 24 may be used to grasp and retrieve stones or other objects toward the distal portion of the guide (424). As the retrieved object approaches the guide (424), energy source (17036, 16026) breaks up the object in the basket tool (2402); this operation is similar to the operation in the embodiment illustrated in FIG. 18.

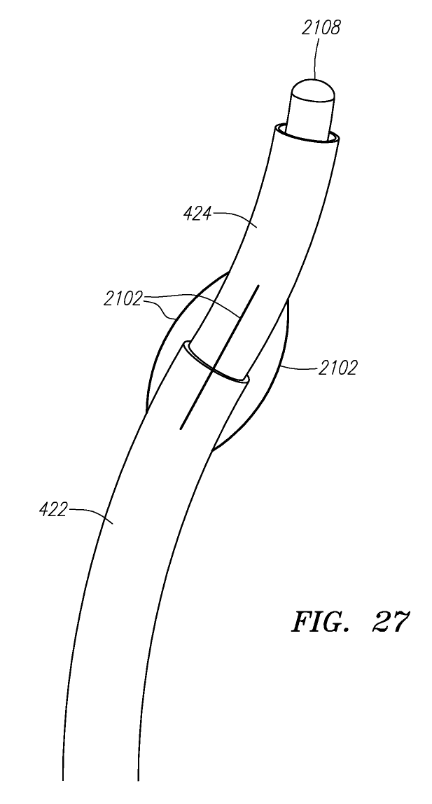

FIG. 27 depicts an embodiment with a proximal basket arm capture (2102) and an image capture device (2108). As described above in the portion of the description describing FIG. 21, when an object is observed with the image capture device (2108), the entire assembly may be advanced while the guide instrument (424) is withdrawn proximally into the sheath instrument (422) until the depicted basket capture arms (2102) are able to rotate toward the central axis of the guide instrument (424) working lumen and capture objects positioned adjacent the distal tip of the guide instrument (424).

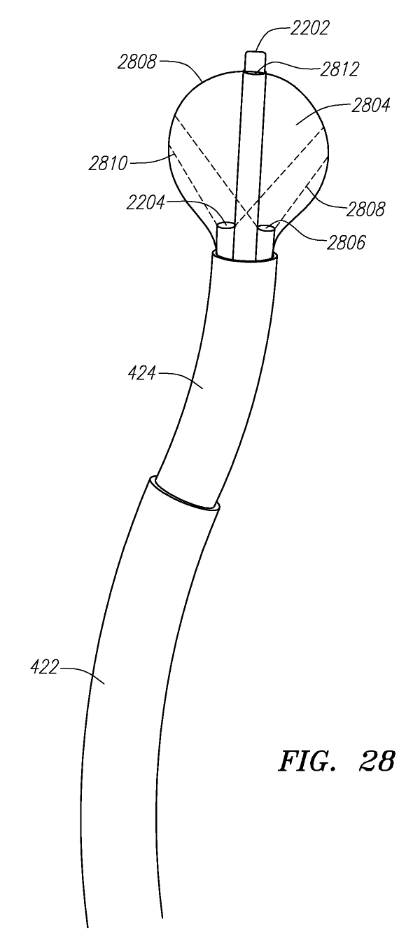

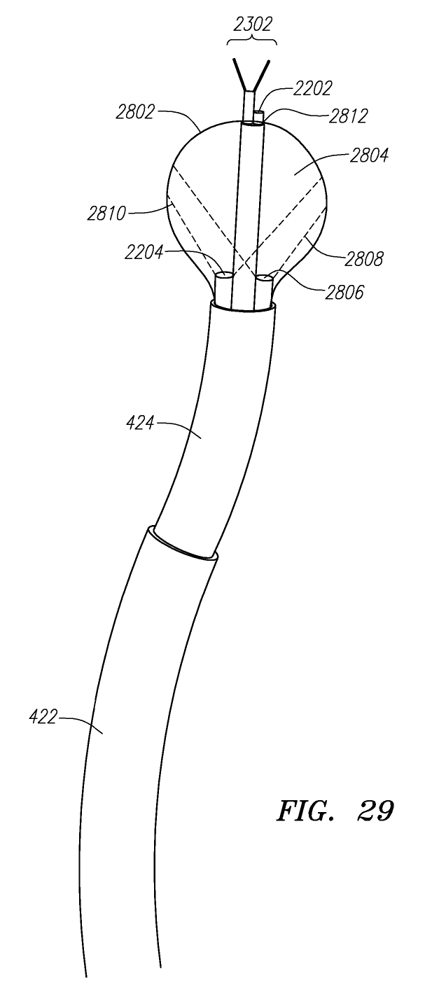

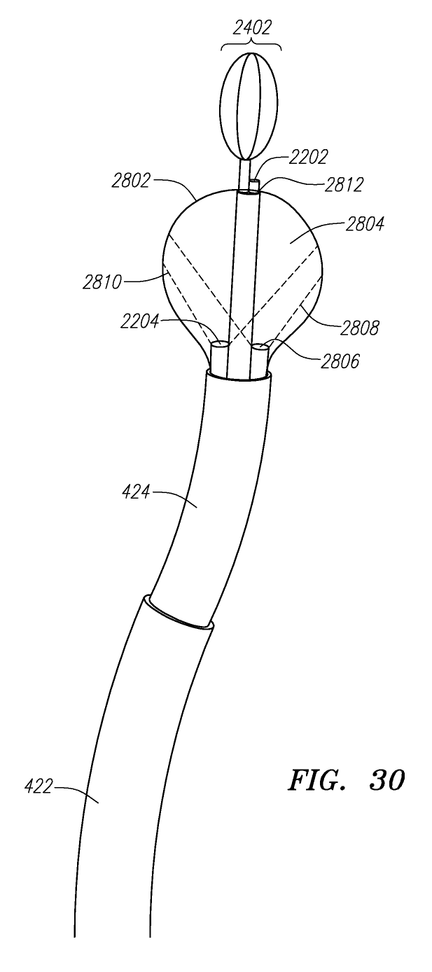

FIG. 28 depicts a configuration with an inflatable balloon (2802) configured to be controllably filled with or evacuated of saline (2804), through which an image capture device (2204) and illumination source (2806) may be utilized to observe objects forward of the balloon that preferably fall within the field of broadcast (2808) of the illumination source (2806) and field of view (2810) of the image capture device (2204). The balloon (2802) also defines a working lumen (2812) through which various tools may be passed—such as a laser fiber (2202), as depicted. FIG. 29 depicts a similar embodiment also comprising a grasping tool (2302). FIG. 30 depicts a similar embodiment with a basket tool (2402).

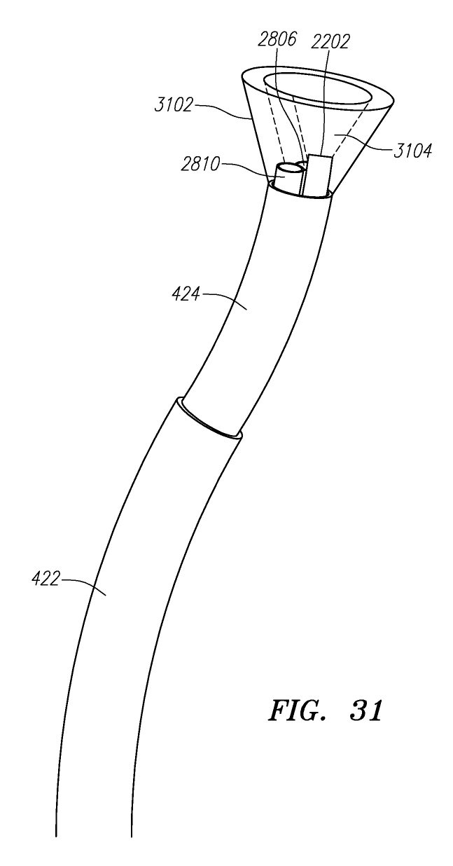

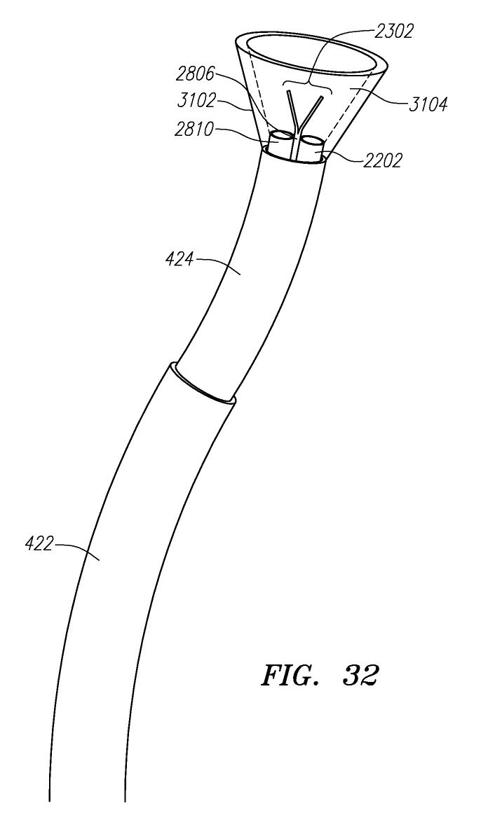

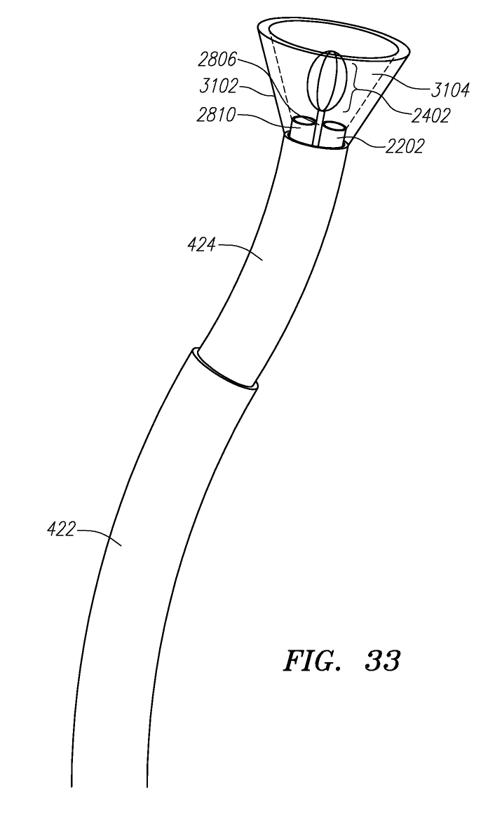

FIG. 31 through FIG. 33 depict similar embodiments which comprise an inflatable balloon cuff (3102) configured to provide a distal working volume (3104) which may be flushed with a saline flush port (2806). The inflatable balloon cuff (3102) preferably works not only as an atraumatic tip, but also as a means for keeping the image capture device (2810) positioned slightly proximally of structures that the inflatable balloon cuff (3102) may find itself against—thus providing a small amount of volume to image such structures without being immediately adjacent to them. With an optical fiberscope as an image capture device (2810), it may be highly valuable to maintain a translucent saline-flushed working volume (3104) through which the image capture device (2810) may be utilized to image the activity of objects, such as tissues and/or kidney stones, as well as the relative positioning of tools, such as fibers, graspers, baskets, etc., from proximal positions into the working volume (3104)—which may be used, for example, to grasp and/or modify or destroy stones or other structures. The inflatable balloon cuff (3102) may be advanced to the desired operational theater, such as the calices of a kidney, in an uninflated configuration, and then inflated in situ to provide the above functionality. Alternatively, the cuff (3102) may be inflated before completing the navigation to the operational theater, to provide atraumatic tip functionality as well as image capture guidance and deflection from adjacent objects, during navigation to the desired operational theater.

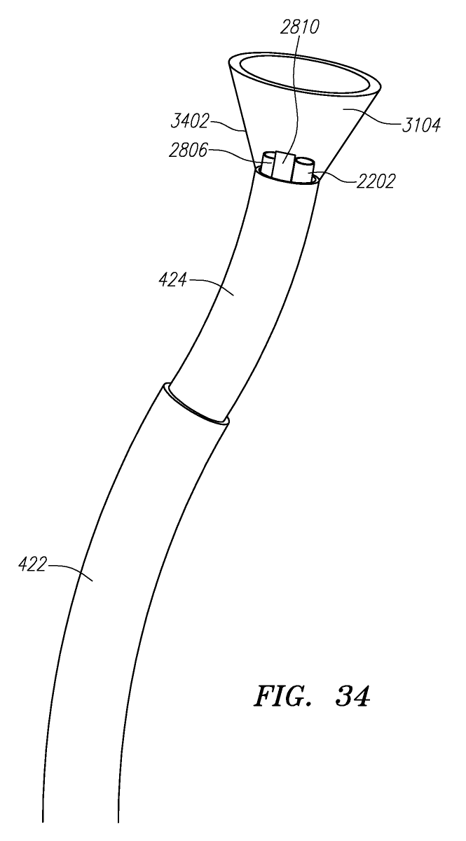

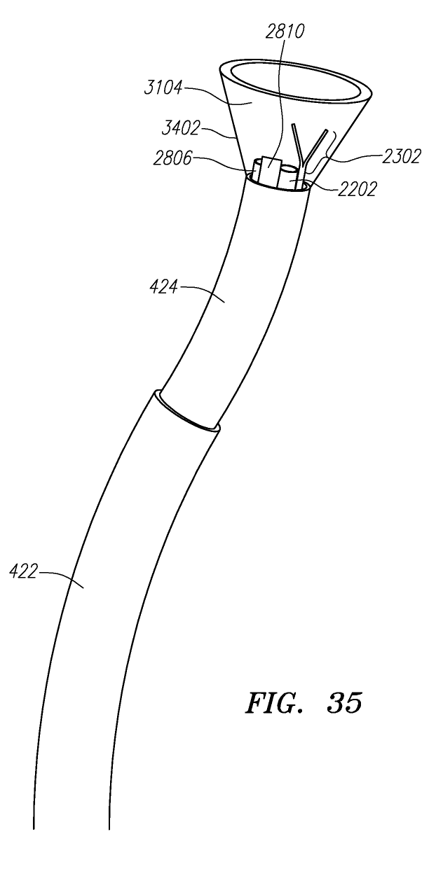

FIG. 34 through FIG. 36 depict similar embodiments, but with a flexible cuff (3402), preferably comprising a soft polymer material, rather than an inflatable cuff (3102) as in the previous set of figures. The flexible cuff (3402) is configured to have similar functionalities as those described in reference to the inflatable cuff (3102) above.

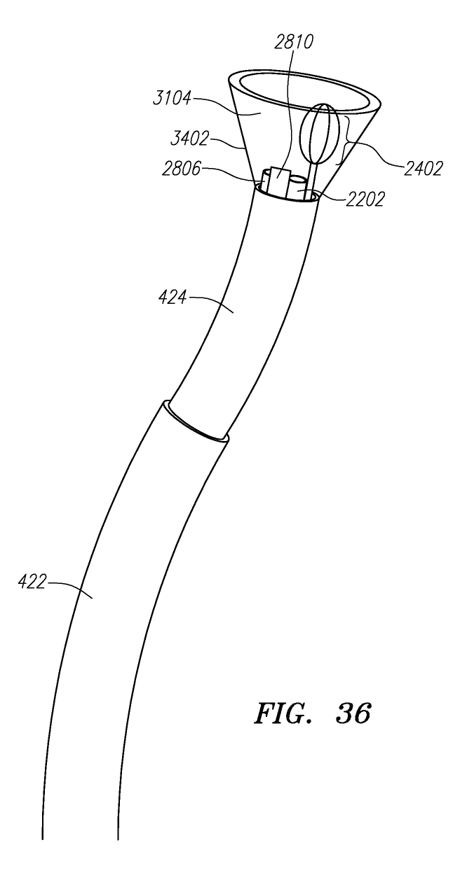

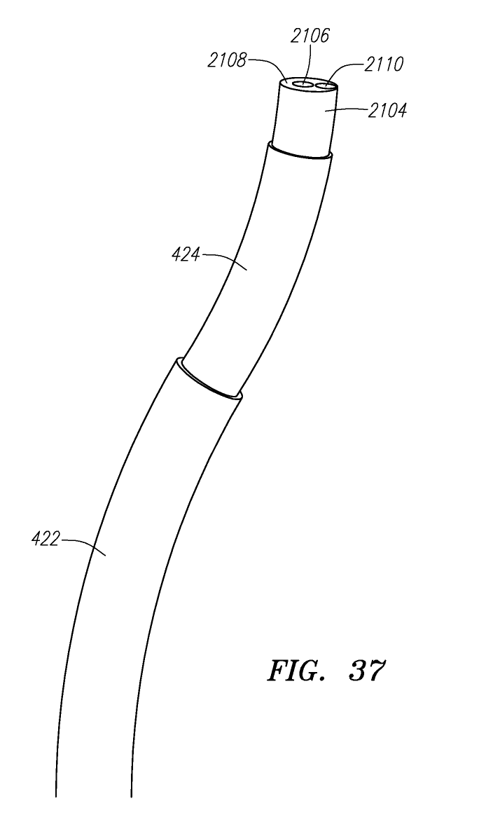

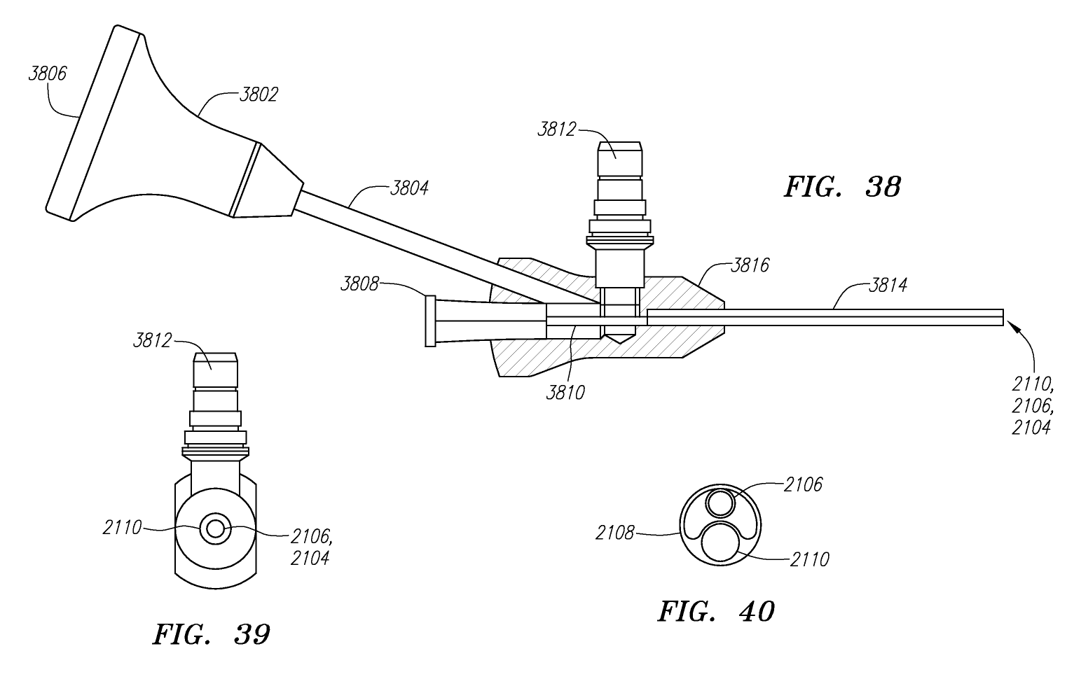

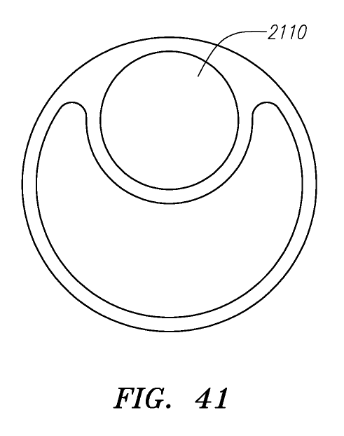

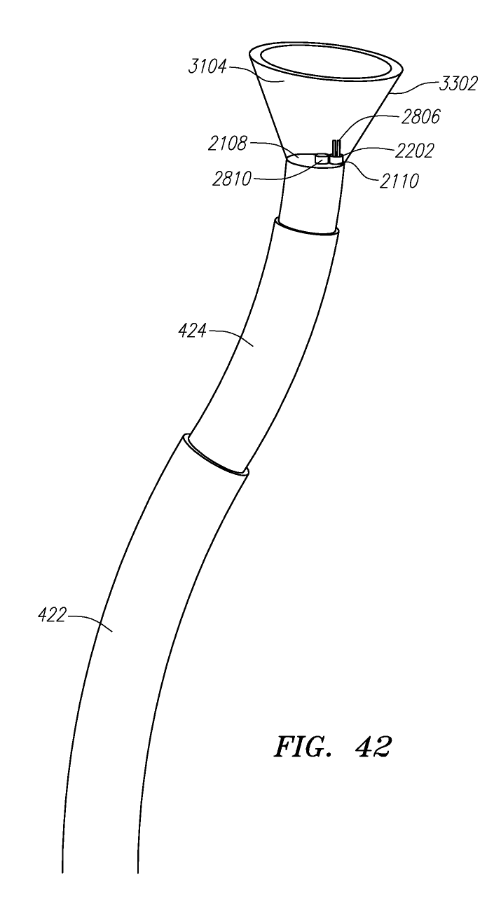

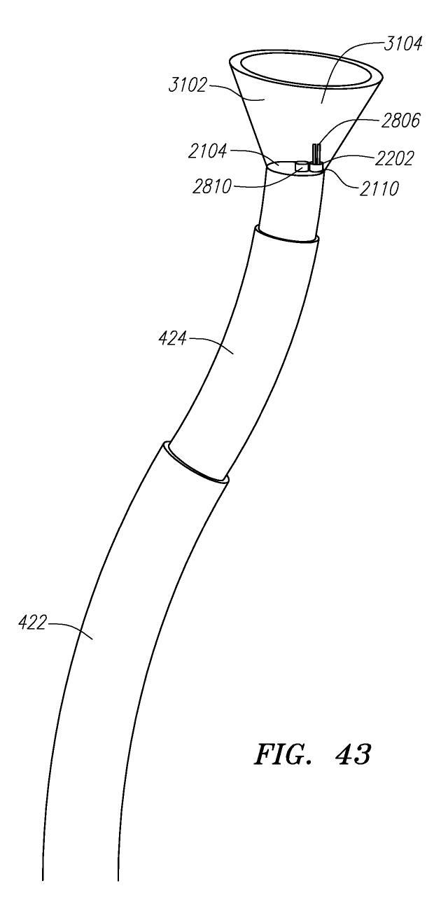

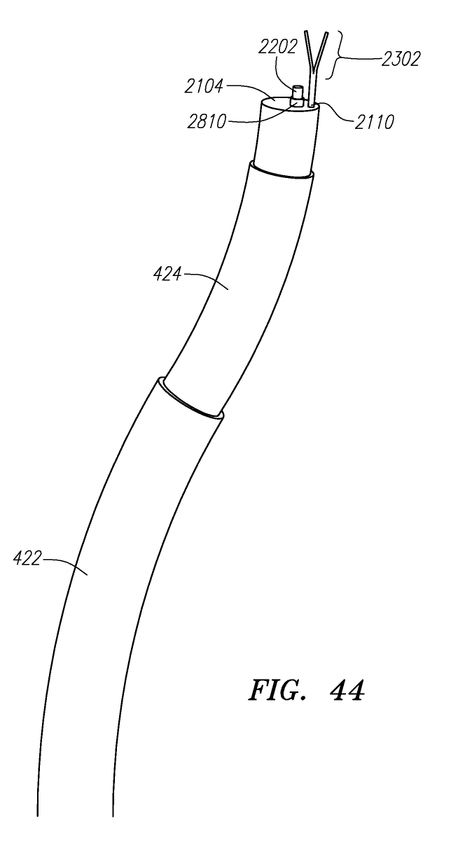

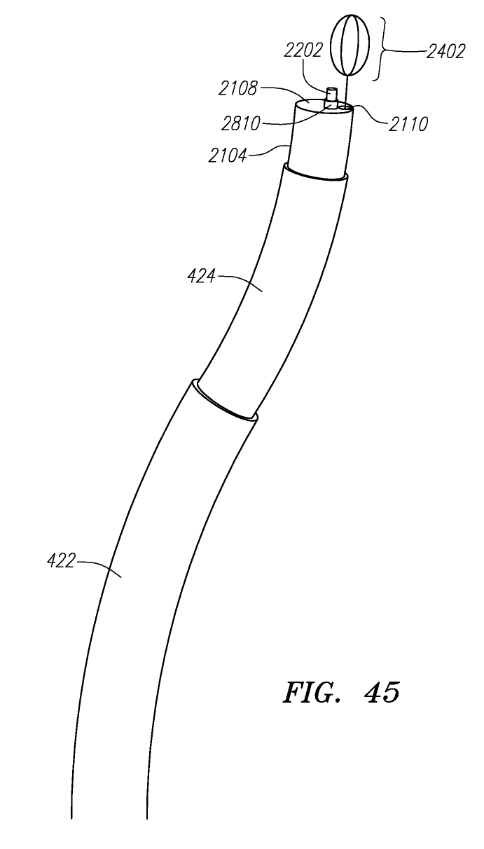

FIG. 37 through FIG. 41 depict an embodiment wherein an assembly of an image capture device (2104), which may optionally comprise a lens (2106), transmission fibers (2108) for imaging, and a working lumen (2110), through which various tools or combinations of tools may be positioned. The components of this embodiment are all packaged within one tubular structure as illustrated in the cross sectional view of FIG. 41, which may comprise a co-extruded polymeric construct. FIG. 38 through FIG. 40 depict the interconnectivity of an image capture device (2104), such as a fiberscope comprising a proximal optics fitting (3802), an optics body member (3804), a proximal surface (3806) for interfacing with a camera device with the illumination fibers and working lumen, comprising a female luer fitting (3808) for accessing the working lumen (2110), a working lumen proximal member (3810), an illumination input tower (3812), an insertion portion (3814), a central body structure (3816). Variations of this embodiment are depicted in FIG. 42 through FIG. 45, with different distal configurations similar to those depicted in reference to the figures described above. FIG. 42 depicts a variation having a distally-disposed flexible cuff (3402) defining a working volume (3104) flushable with a saline port (2806) and imaged with an image capture device (2810) as described above. FIG. 43 depicts a similar variation having an inflatable cuff (3102). Tools such as graspers, energy sources, fibers, baskets, etc may be utilized through the working lumens (2110) of the embodiments depicted in FIG. 42, FIG. 43, FIG. 44, FIG. 45, etc. The embodiment of FIG. 44 comprises a grasping tool (2302) positioned through the working lumen of the assembly (2104—the assembly depicted in FIG. 37 through FIG. 41), which the embodiment of FIG. 45 comprises a basket tool (2402).

Each of the above discussed tools, configurations, and/or assemblies may be utilized for, among other things, endolumenal urinary intervention, such as the examination, removal, fragmentation, and/or destruction of stones such as kidney or bladder stones.

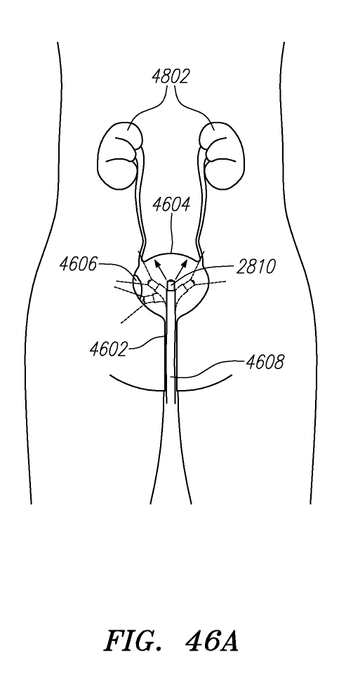

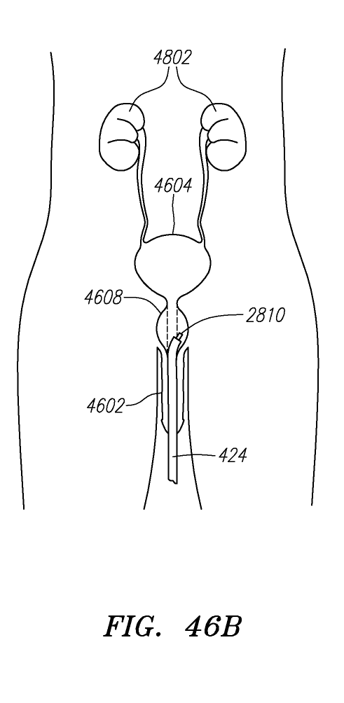

Referring to FIG. 46A, a steerable instrument assembly according to one embodiment may be steered through the urethra (4602) and into the bladder (4604), where an image capture device (2810) may be utilized, as facilitated by injected saline, to conduct a cystoscopy and potentially observe lesions (4606) of interest. The omni-directional steerability and precision of the robotic guide and/or sheath to which the image capture device is coupled facilitates collection of images of inside of the bladder (4606) which may be patched together to form a 3-dimensional image. The instrument assembly (108-422, 424, 2810) may also be utilized to advance toward and zoom the image capture device upon any defects, such as obvious bleeds or tissue irregularities. Indeed, aspects of the images captured utilizing the image capture device (2810) may be utilized in the controls analysis of the subject robotic catheter system to automate, or partially automate aspects of the system/tissue interaction. For example, as described above, more than one two-dimensional image may be oriented relative to each other in space to provide a three-dimensional mosaic type composite image of a subject tissue mass, instrument, or the like. Localization techniques may be utilized to assist with the “gluing together” of more than one image; for example, spatial coordinates and orientation may be associated with each image captured by the image capture device, to enable re-assembly of the images relative to each other in space. Such a three-dimensional composite image may be registered in three dimensions to the workspace or coordinate system of the subject elongate instrument or instrument assembly, to provide automated display, zooming, and reorientation of the images displayed relative to the distal portion of the elongate instruments as the instruments are moved around in the workspace. Further, the system may be configured to update the composite image with more recently-captured images as the instruments are navigated about in the workspace. Image recognition algorithms may be utilized to bolster the information gleaned from image capture; for example, a substantially round and dark shape in a particular location known to be at least relatively close to a lumen entry into or exit from a particular anatomic space may be analyzed and determined via application of the pertinent algorithms to be a given lumen entry or exit anatomical landmark, and the location of such landmark may be stored on a database along with the position and orientation variables of the elongate instruments utilized in the particular instance to arrive at such location—to enable easy return to such location using such variables. The system may thus be configured to allow for automated return of the instruments to a given landmark or other marker created manually or automatically upon the composite image and associated database. Further, given the composite image of the actual tissue in-situ, the system may be configured to not only to allow for the storage of and return to certain points, but also for the creation and execution of configurable “keep out zones”, into which the instruments may be disallowed under navigation logic which may be configured to prevent touching of the instruments to certain tissue locations, navigation of the instruments into particular regions, etc. Similar procedures may be performed in the prostate (4608) as illustrated in FIG. 46B.

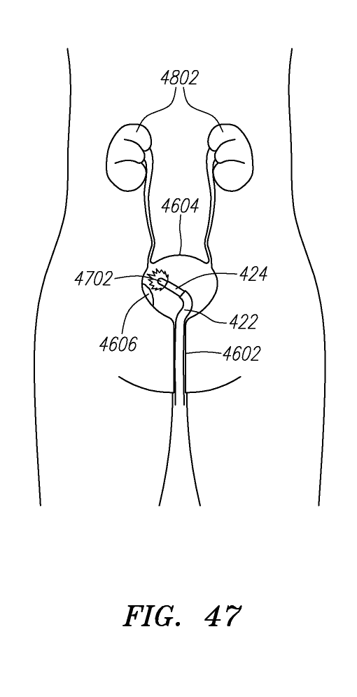

Referring to FIG. 47, the instrument assembly (108-422, 424, 4702) may alternatively or additional comprise an interventional tool such as an ablation tool (4702) for ablating tumors or other lesions (4606) within the bladder (4604) or prostate (4608). Any of the above-discussed assemblies may be utilized for such a cystoscopy procedure.

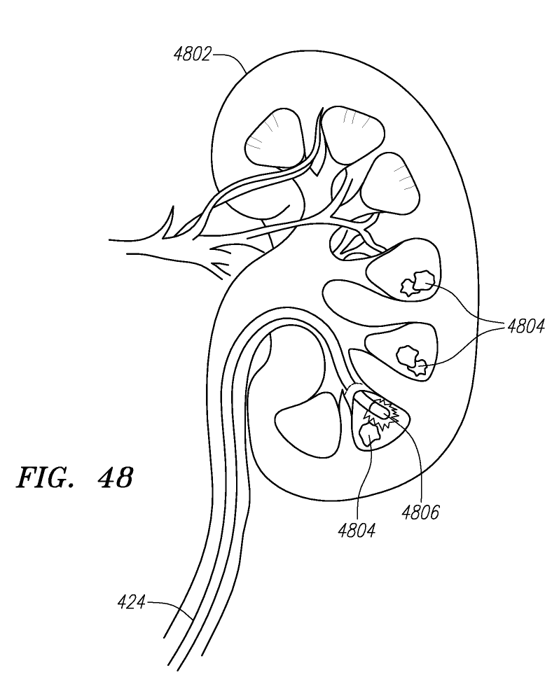

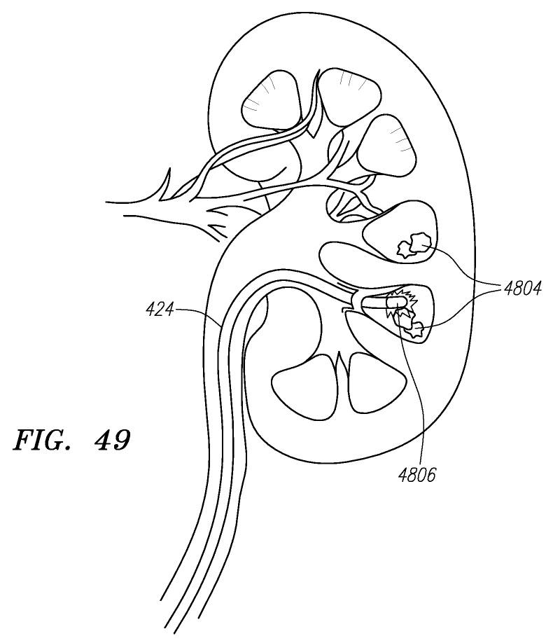

Each of the above-discussed constructs may also be utilized adjacent to or within the kidneys. Referring to FIG. 48 and FIG. 49, for illustrative purposes, a portion of a relatively simple instrument assembly embodiment (for example, a sheath distal tip may be positioned in the bladder at the entrance to the urethra while the more slender guide, 424, is driven toward and into the kidney, 4802) is depicted. Such assembly may be advanced toward and/or steerably driven into the kidney (4802), where stones (4804) may be captured with graspers or other tools, or where stones may be destroyed using chemistry, cryo, RF, laser lithotripsy, or laser ablation tools (4806), or other radiative techniques, such as ultrasound, as depicted in FIG. 48 and FIG. 49. Each of the tools, configurations, and/or assemblies discussed above in reference to FIG. 16 through FIG. 45 may be utilized for the examination, removal, fragmentation, and/or destruction of stones such as kidney or bladder stones. Preferably, an image capture device (2810) is positioned in or adjacent to the calices of the kidney to enable interactive viewing of objects such as stones, while various tool configurations may be utilized to examine, capture, grasp, crush, remove, destroy, etc, such stones, before withdrawing the instrument assembly.

Certain control system paradigms developed for more conventional robotic systems, such as rigid instrument robotic systems, are not entirely applicable to a flexible robotic platform such as those described herein. One the key differences is that instrument configurations such as the flexible robotic catheter assemblies (e.g., 108, 422, and along with various operatively coupled tools such as 16026, 17026, 17036, 18026, 19024, 2102, 2104, 2202, 2204, 2302, 2402, etc.) depicted in FIG. 16 through FIG. 45 are configured to be compliant for anatomical, safety, and other reasons, as opposed to rigid and/or back-drivable systems, for example. Indeed, with certain embodiments of the present invention, contact from adjacent soft tissue structures may produce forces large enough to push the instrument assembly off of the predicted navigation trajectory or even cause one or both instruments to become temporarily stuck in a particular position. To accommodate the compliance of such instrument embodiments, it may be desirable to control for factors other than simple instrument tip position. For example, in one embodiment, it is desirable to control at least one axis of a distal tip coordinate system for velocity rather than position. In one variation of such embodiment, up-down and left-right may be controlled conventionally for position, while insertion-retraction of the instrument may be controlled for velocity—somewhat in the manner in which a submarine might be controlled—to make the experience of navigating with a forward-oriented real-time image capture device in an embodiment such as that depicted in FIG. 37 through FIG. 41 as simple and instinctive as possible. This may be accomplished, for example, with a separate input device for velocity-controlled insertion-retraction and a separate input device for up-down and left-right, or with a single input device.

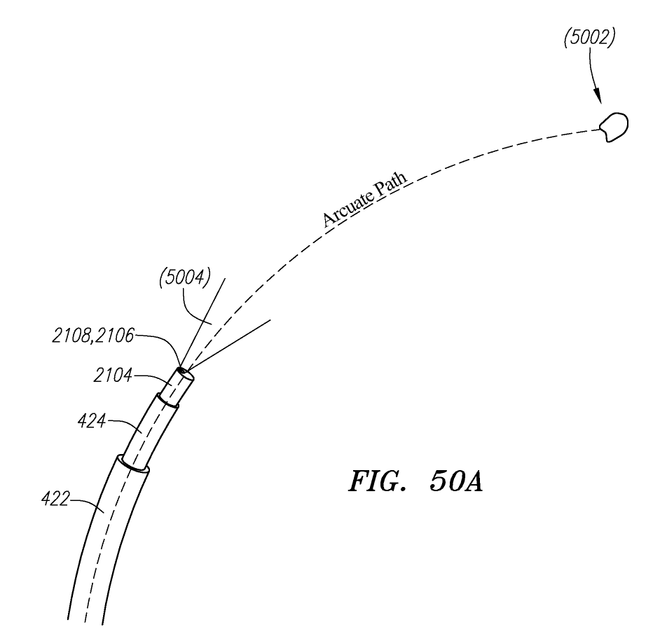

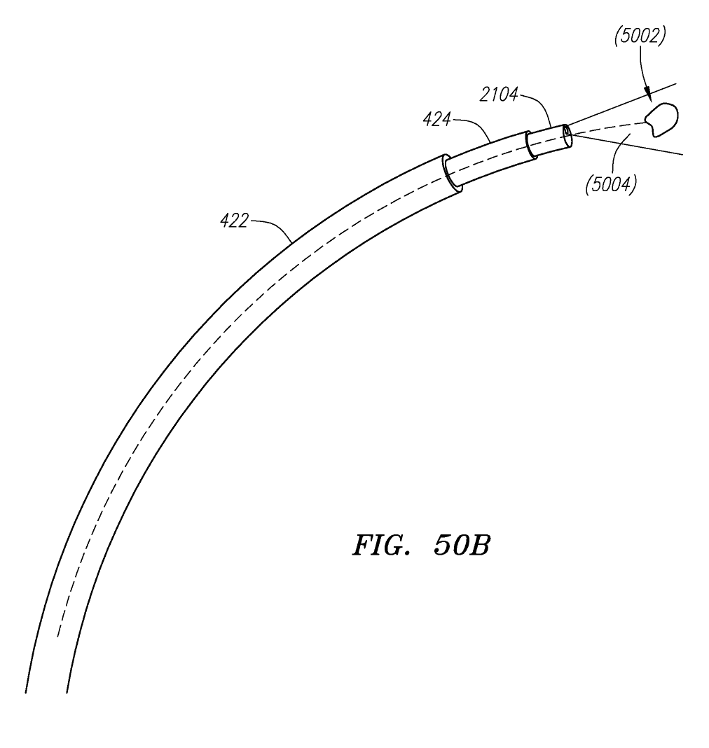

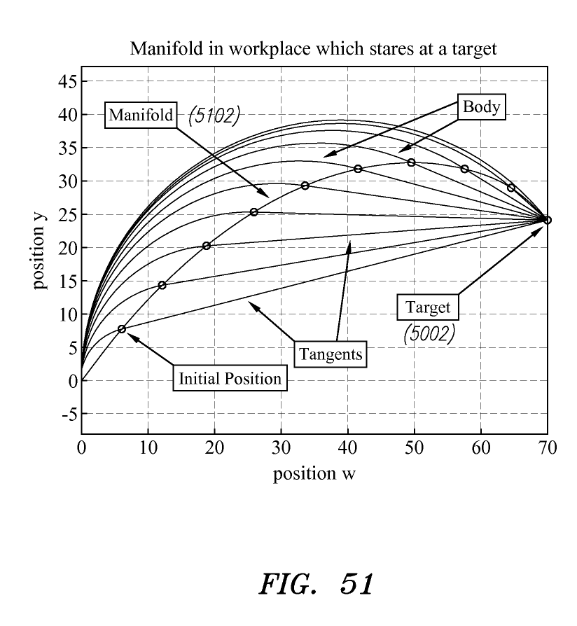

In another embodiment, it may be desirable to control for forward-oriented field of view orientation and/or position rather than instrument position and/or orientation. Referring to FIG. 50A, an instrument assembly (108) such as that depicted in FIG. 37 through 41 is depicted as it is being navigated toward a target object (5002), such as a kidney stone or tissue lesion. In such scenario, it is desirable to keep the target within the forward-oriented field of view (“FOV”) (5004) of the image capture device (2104) that includes lens (2106) and transmission fibers (2108), which in this embodiment, is aligned with the distal portion of the instrument assembly. With the target (5002) in the FOV (5004), the operator (116) may use the controls interface, e.g., (118) or (120), to advance the instrument assembly (108) toward the target (5002) while keeping the target (5002) in view. In one scenario, the operator (116) may attempt to destroy or alter the target (5002) using, for example, a laser lithotripsy fiber (16026). In other scenario, the operator (116) may capture the target with a basket apparatus or manipulate the target with a tool such as a gripper, etc. In an embodiment wherein the instrument assembly (108) is navigated under position control, or a combination of position control and velocity control for insertion, as described above, the controls algorithms may insert the instrument assembly (108) along an arcuate path toward the target (5002) as per the commands of the operator (116) (i.e., should the operator direct the instrument assembly to move toward the target), as depicted in FIG. 50A, with the FOV (5004) following such arcuate pathway. One disadvantage of this controls scenario is that the FOV (5004) of the image capture device (2104) would follow the arcuate path and may lose sight of the target (5002) during the initial portion of the navigation trajectory, as depicted in FIG. 50A, only to catch up with the target (5002) at the end of the trajectory, as depicted in FIG. 50B. Further, without knowing the position of the target (5002) relative to the starting position of the instrument assembly (108) and image capture device (2104), it may be difficult for the control system to select an efficient trajectory that will end with the target (5002) in the FOV (5004). Referring to FIG. 51, a plot depicts initial position of the forward-oriented image capture device of such a system, the position of the target, and the position of the instrument assembly body orientation required to keep the target in the center (illustrated by tangent lines) of the FOV of the image capture device. Given the position of the target (5002) relative to the position of the image capture device (2104), an instrument assembly body orientation may be determined and utilized by the control system to keep the target in the FOV during advancement of the instrument assembly body along the manifold curve (5102) depicted in FIG. 51. FIG. 51 depicts a series of instrument assembly body positions as the instrument assembly (108) including both the sheath catheter (422) and guide catheter (424) or just the guide catheter (424) is advanced toward the target (5002). The position of the target (5002) relative to the image capture device (2104) may be determined with imaging techniques (for example, ultrasound, localization, preoperative CT scanning, stereoscopic imaging, etc) and subsequent registration with the instrument assembly (108) using, for example, localization sensors or anatomy-based registration and/or calibration techniques. In the case of the calices of the kidney, a preoperative contrast agent injection may be captured with an image capture device and segmented to produce a fairly clean model of the calices for preoperative planning and intra-operative navigation subsequent to registration and/or calibration.

Given a scenario wherein the position of the target (5002) relative to the image capture device (2104) is unknown, receding horizon control algorithms may be utilized, wherein an initial position of the target (5002) relative to the image capture device (2104) is estimated, and the orientation of the distal portion of the instrument assembly (108) is bent toward the target at an angle which may be determined for a given instantaneous relative positioning scenario using a model such as that depicted in FIG. 51. In one embodiment, rather than bending the instrument assembly (108) to bring the FOV (5004) of the image capture device (2104) straight-on the tangent line and potentially risk overshooting the target with too much adjustment, it may be desirable to use slightly less bending—known as a “discounted tangent” scenario—and continue to iterate the orientation of the FOV relative to the target with further discounted tangent adjustment as the instrument assembly is advanced toward the target. Thus, a FOV control scenario may be realized for navigation with an image capture device, as opposed to conventional instrument position control.

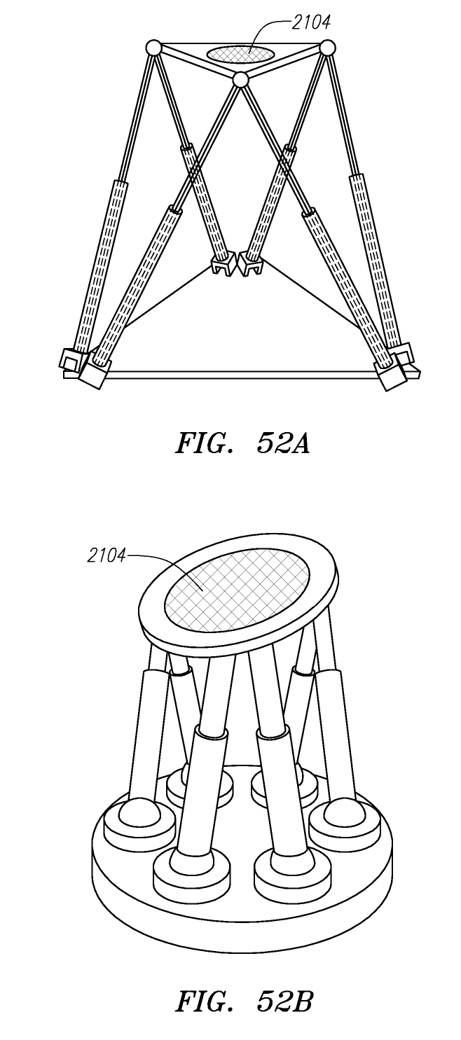

In another embodiment, rather than employing a FOV control scenario such as that described above, it may be desirable to maintain simple position control, or position/velocity control, move along an arcuate or other trajectory, and adjust the captured FOV to keep desired target objects visible during navigation of an instrument or instrument assembly. For example, in one embodiment, an image capture device (2104) such as an optical imaging chip may be coupled to a Stewart or Gough platform mechanism, as depicted in FIG. 52A and FIG. 52B, such platform mechanism being coupled to the instrument assembly (108) and controllable by the operator (116) at the workstation (102) to preferably orient the image capture device (2104) and resultant FOV (5004). In another embodiment, a controllably re-orientable mirror or prism may be utilized for similar result. In another embodiment, a fish-eye type lens could be utilized with a high-resolution image capture device and proximal control system to only capture or present certain sectors of the spectrum of the total image capture from the fish-eye lens, to enable the operator to focus on one particular sub portion of this large FOV.

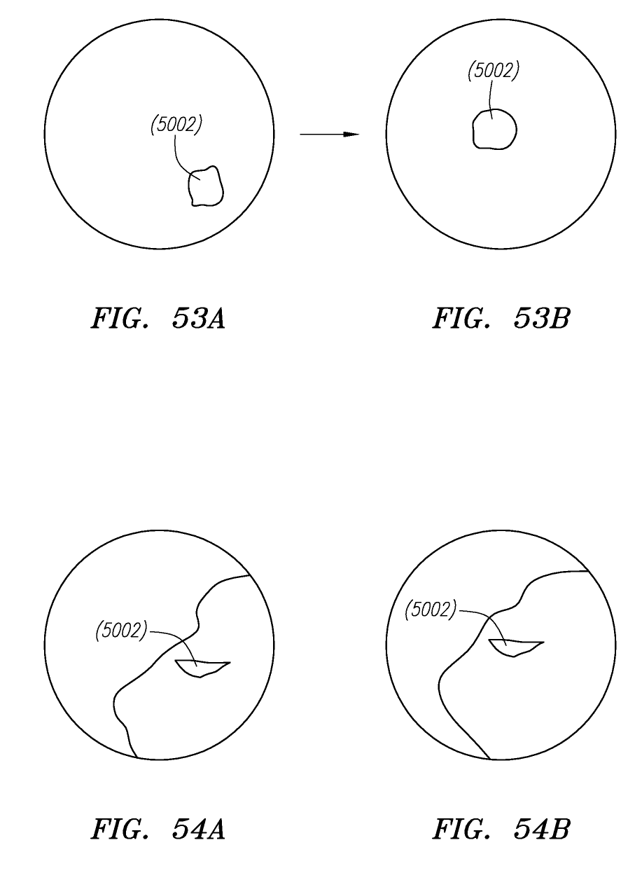

In another embodiment, image processing and pattern recognition techniques may be utilized to keep an identified target object (5002) centered within the presented field of view, as depicted in the correction from FIG. 53A to FIG. 53B. Similarly, image processing and pattern recognition techniques may be utilized to keep an identified portion of an object (5002), such as an irregularity, margin, or aperture, centered within the presented field of view (5004), as depicted in the correction from FIG. 54A to FIG. 54B.

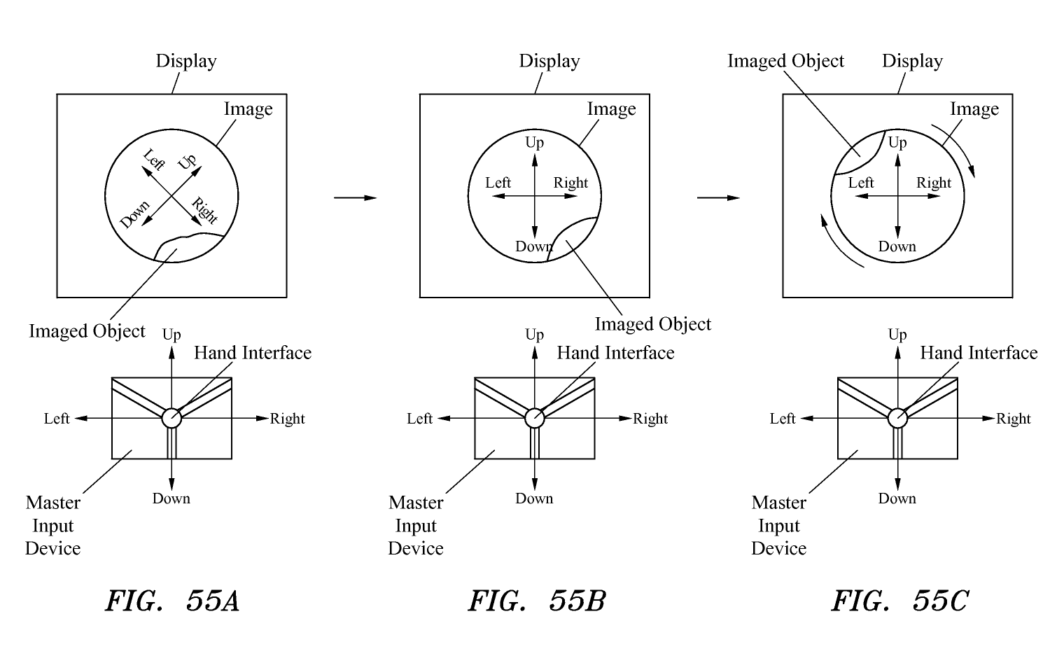

Referring to FIG. 55A, it may be desirable to calibrate master input device orientation with views presented on the associated display for maximum simplicity and instinctiveness of control by the operator. In the event the a straight up command to the master input device from the operator through the hand interface with an embodiment such as that depicted in FIG. 37 through FIG. 41 wherein image-based navigation is desired, it is preferable to have the instrument assembly move the FOV of the image capture device straight up in response to such straight up command at the master input device. In other words, it is desirable to have “up” at the master means “up” with the FOV. FIG. 55A depicts a scenario wherein the coordination of the FOV movement and master input device movement is approximately 45 degrees out of sync. In one embodiment, the system is configured to allow the operator to recalibrate the synchronization of movement between the instrument assembly with associated image capture device and the master input device by switching to a calibration mode wherein the operator reconfigures the associated transformations to associate the master and presented FOV as depicted in FIG. 55B—with “up” on the master being “up” with the FOV, “left” as “left”, “right” as “right”, “down” as “down”, “clockwise rotation” as “clockwise rotation”, “counter-clockwise rotation” as counter-clockwise rotation”, etc. In another variation, as depicted in FIG. 55C, subsequent to calibration as described in reference to FIG. 55A to FIG. 55B, the operator may wish to reorient the FOV image presented at the display (for ease of control, familiarity, etc reasons) and have all of the coordination/calibration between the master and FOV movement remain coordinated (“up” at the master still being “up” with the FOV as displayed, without regard to the new orientation of the image rotationally relative to the display).

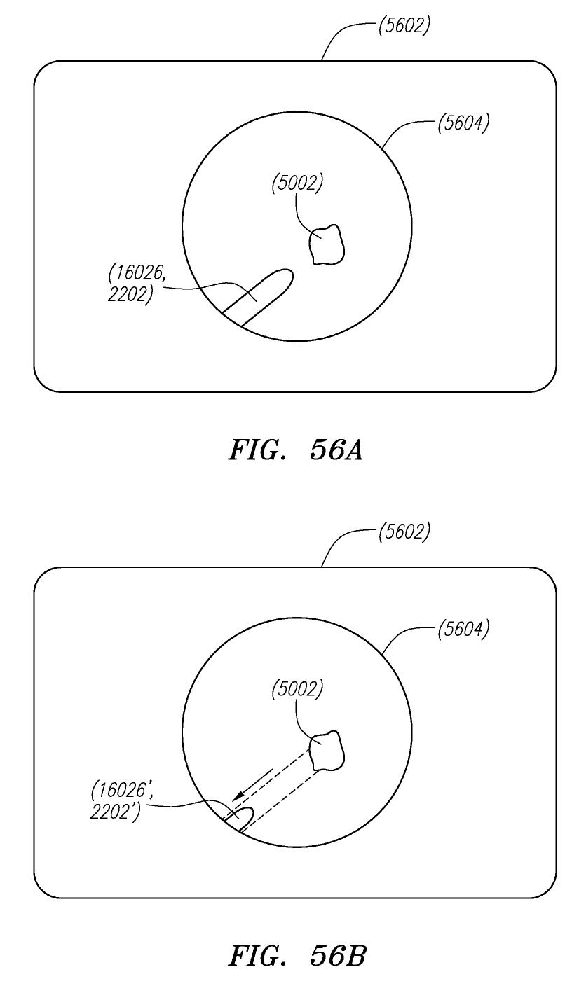

Referring to FIG. 56A, when a target (5002) has been approached in adequate proximity for intervention with instrumentation or tools such as a laser lithotripsy fiber (e.g., 16026, 2202, etc.), it is desirable to also have the FOV capturing a portion of the subject instrument or tool. For example, a monitor (122) may provide a display (5602) of an image (5604) that is captured by an image capture device (2104) showing the relative positions of the target (5002) and a laser fiber. Alternatively, should the instrument or tool be outside of the FOV (5004), e.g., the laser fiber might be withdrawn proximally into a lumen of the instrument assembly or otherwise not within the FOV, it is preferable to present to the operator an indication of the position and/or orientation of the laser fiber or any of such instrument or tool relative to the FOV, as depicted in the embodiment of FIG. 56B, to enable the operator to have expectations regarding where the instrument or tool will indeed enter the FOV should he advance it, etc.

While multiple embodiments and variations of the many aspects of the invention have been disclosed and described herein, such disclosure is provided for purposes of illustration only. Many combinations and permutations of the disclosed system, apparatus, and methods are useful in minimally invasive medical diagnosis and intervention, and the invention is configured to be flexible and adaptable. The foregoing illustrated and described embodiments of the invention are suitable for various modifications and alternative forms, and it should be understood that the invention generally, as well as the specific embodiments described herein, are not limited to the particular forms or methods disclosed, but also cover all modifications, alternatives, and equivalents as defined by the scope of the appended claims. Further, the various features and aspects of the illustrated embodiments may be incorporated into other embodiments, even if not so described herein, as will be apparent to those skilled in the art. All directional references (e.g., upper, lower, upward, downward, left, right, leftward, rightward, top, bottom, above, below, vertical, horizontal, clockwise, counterclockwise, etc.) are only used for identification purposes to aid the reader's understanding of the invention without introducing limitations as to the position, orientation, or applications of the invention. Joining references (e.g., attached, coupled, connected, and the like) are to be construed broadly and may include intermediate members between a connection of elements (e.g., physically, electrically, optically as by an optically fiber, and/or wirelessly connected) and relative physical movements, electrical signals, optical signals, and/or wireless signals transmitted between elements. Accordingly, joining references do not necessarily infer that two elements are directly connected in fixed relation to each other. It is intended that all matters contained in the description or shown in the accompanying drawings shall be interpreted as illustrative only and not limiting. Modifications, alternatives, and equivalents in the details, structures, or methodologies may be made without departing from the scope of the invention as defined by the appended claims.

What is claimed is:

1. A robotic surgical system, comprising:
an instrument driver;
an instrument assembly operatively coupled to the instrument driver such that mechanisms of the instrument driver operate or control movement, operation, or both, of components of the instrument assembly, the instrument assembly components including an elongate flexible guide instrument and an image capture device, wherein the image capture device is configured to capture images of a forward-oriented field of view; and
a controller operatively coupled to the instrument driver and configured to operate the instrument driver mechanisms in a manner so as to control advancement of the instrument assembly toward a target along a trajectory that maintains the target in the forward-oriented field of view of the image capture device.

2. The robotic surgical system of claim 1, wherein the controller utilizes a software-implemented orientation platform to maintain the target in the forward-oriented field of view of the image capture device.

3. The robotic surgical system of claim 2, wherein the orientation platform is a Stewart or Gough platform.

4. The robotic surgical system of claim 1, wherein the controller utilizes a software-implemented receding-horizon control algorithm that provides outputs for operating the instrument driver mechanisms to maintain the target in the forward-oriented field of view of the image capture device.

5. The robotic surgical system of claim 1, wherein the controller utilizes a software-implemented pattern recognition algorithm for identifying target objects or target features in images acquired by the image capture device and providing outputs for operating the instrument driver mechanisms to maintain the identified target objects or target features in the forward-oriented field of view of the image capture device.

6. The robotic surgical system of claim 1, wherein the controller is configured to position or orient the elongate flexible guide instrument using discounted tangent adjustments in order to maintain the target in the forward-oriented field of view of the image capture device.

7. The robotic surgical system of claim 1, further comprising a monitor for displaying images of the forward-oriented field of view acquired by the image capture device.

8. The robotic surgical system of claim 7, further comprising an user input device coupled to the controller for controlling movement, operation, or both, of the components of the instrument assembly wherein movement of the user input device is calibrated with the elongate flexible guide instrument such that a directional input to the user input device produces a corresponding directional movement of the forward-oriented field of view displayed on the monitor.

9. The robotic surgical system of claim 7, wherein the controller is operatively coupled to the display and configured to supply an indicated image of a working tool on the display when the working tool is outside of the forward-oriented field of view.

10. The robotic surgical system of claim 1, further comprising a working tool operatively coupled to the instrument assembly and configured to be independently navigated relative to the guide instrument.

11. The robotic surgical system of claim 9, wherein the working tool is selected from the group comprising
a laser fiber,
a gripper, and
a basket.

12. The robotic surgical system of claim 1, wherein the image capture device includes a fish-eye type lens for capturing or presenting selected sectors of the forward-oriented field of view.

13. The robotic surgical system of claim 1, wherein the controller is operatively coupled to the instrument driver via a remote communication link.

14. A robotic surgical system, comprising:
an instrument driver;
an instrument assembly operatively coupled to the instrument driver such that mechanisms of the instrument driver operate or control movement, operation, or both, of components of the instrument assembly, the instrument assembly components including an elongate flexible guide instrument and an image capture device, wherein the image capture device is configured to capture images of a forward-oriented field of view; and
a controller operatively coupled to the instrument driver, wherein the controller utilizes a software-implemented pattern recognition algorithm for identifying target objects or target features in images acquired by the image capture device, and wherein the controller is further configured for operating the instrument driver mechanisms so as to control movement of the guide instrument while maintaining the identified target objects or target features in the forward-oriented field of view of the image capture device.

15. The robotic surgical system of claim 14, wherein the controller is configured to position or orient the elongate flexible guide instrument using discounted tangent adjustments in order to maintain the identified target objects or target features in the forward-oriented field of view of the image capture device.

16. The robotic surgical system of claim 14, further comprising a monitor for displaying images of the forward-oriented field of view acquired by the image capture device.

17. The robotic surgical system of claim 16, further comprising an user input device coupled to the controller for controlling movement, operation, or both, of the components of the instrument assembly wherein movement of the user input device is calibrated with the elongate flexible guide instrument such that a directional input to the user input device produces a corresponding directional movement of the forward-oriented field of view displayed on the monitor.

18. The robotic surgical system of claim 7, wherein the controller is operatively coupled to the display and configured to supply an indicated image of a working tool on the display when the working tool is outside of the forward-oriented field of view.
